(19) United States
(12) Patent Application Publication
(10) Pub. No.: US 2024/0145032 A1
(43) Pub. Date: May 2, 2024

(54) METHODS AND GENOMIC CLASSIFIERS FOR PROGNOSIS OF BREAST CANCER AND IDENTIFYING SUBJECTS NOT LIKELY TO BENEFIT FROM RADIOTHERAPY

(71) Applicant: PFS Genomics, Inc., Madison, WI (US)

(72) Inventors: S. Laura Chang, Verona, WI (US); Lori J. Pierce, Ann Arbor, MI (US); Corey Speers, Dexter, MI (US); Felix Feng, Hillsborough, CA (US); Per Malmström, Skanör (SE); Mårten Fernö, Limhamn (SE); Erik Holmberg, Asperö (SE); Per O. Karlsson, Gothenburg (SE)

(21) Appl. No.: 18/548,395

(22) PCT Filed: Mar. 1, 2022
(86) PCT No.: PCT/US22/18313

§ 371 Date: Aug. 30, 2023

Related U.S. Application Data
(60) Provisional application No. 63/154,821, filed on Mar. 1, 2021.

(51) Int. Cl. G16B 20/00; G16B 25/10; G16B 40/20; G16H 50/20

(52) U.S. Cl. CPC G16B 20/00; G16B 25/10; G16B 40/20; G16H 50/20

(57) ABSTRACT

The present disclosure relates to systems and methods for providing individualized prognostic assessments of breast cancer recurrence (e.g., locoregional recurrence). The systems and methods involve measuring gene expression from a patient sample to create a gene expression signature which identifies subjects who are not likely to benefit from radiotherapy following breast cancer surgery.

### CROSS-REFERENCE TO RELATED APPLICATION

This application claims priority to U.S. Provisional Patent Application Ser. No. 63/154,821, filed on Mar. 1, 2021, which is hereby incorporated by reference herein in its entirety.

### FIELD OF THE INVENTION

The present disclosure relates to systems and methods for providing individualized prognostic assessments of breast cancer recurrence (e.g., locoregional recurrence). The systems and methods involve measuring gene expression from a patient sample to create a gene expression signature which identifies subjects who are not likely to benefit from radiotherapy following breast cancer surgery.

### BACKGROUND OF THE INVENTION

Despite advances in the treatment and diagnosis of breast cancer, there remains a need for improved prognostic assessments of breast cancer recurrence (e.g., locoregional recurrence). Further, there is a need for novel gene signatures that are prognostic for locoregional recurrence and radiation sensitivity.

The present invention addresses these needs.

### SUMMARY OF THE INVENTION

The present disclosure relates to systems and methods for providing individualized prognostic assessments of breast cancer (BC) recurrence (e.g., locoregional recurrence (LRR)). The systems and methods involve measuring gene expression from a patient sample to create a gene expression signature which identifies subjects who are not likely to benefit from radiotherapy (RT) following breast cancer surgery (BCS).

The present disclosure relates to methods, systems, and kits for the diagnosis, prognosis, and treatment of BC in a subject. The disclosure also provides biomarkers and classifiers for identifying subjects at low risk of breast cancer recurrence and not likely to benefit from adjuvant radiotherapy. Further disclosed herein, in certain instances, are probe sets for use in detecting such biomarkers for determining the risk of breast cancer recurrence in a subject. The disclosure further provides biomarkers and classifiers for identifying subjects at risk for locoregional recurrence (LRR) and predicting response to radiotherapy. Methods of treating breast cancer based on expression profiling and/or age to determine the risk of breast cancer recurrence are also provided.

In certain embodiments, the present invention provides methods, comprising: a) measuring an expression level of one or more genes in a biological sample from a human patient having or at risk of having breast cancer (BC), wherein the one or more genes are selected from AGR2, B4GALT1, CLDN7, EZR, GNG11, JUN, MMP11, PKIB, PRPS1, PSMD10, SH3BP5, SLC16A3, SLC7A11, SPP1, TNNT1, and UBE2E1; and b) determining a likelihood of BC recurrence for the patient based on the expression level of the one or more genes selected. In certain embodiments, the method may additionally comprise comparing the measured expression levels of the one or more genes selected to that of pre-determined gene expression levels consistent with risk for BC recurrence or may comprise normalizing the expression levels of the one or more genes selected, for instance, to produce a normalized expression level for the one or more genes selected. In further embodiments, determining the likelihood of BC recurrence may be based on the compared expression level of the one or more genes selected. In another embodiment, determining the likelihood of BC recurrence may be based on the normalized expression level for the one or more genes selected.

In certain embodiments, the disclosure provides methods for predicting a likelihood of recurrence of BC for a patient having BC or at risk of having BC comprising: (a) measuring, in a sample obtained from the patient, an expression level of one or more of the following genes: AGR2, B4GALT1, CLDN7, EZR, GNG11, JUN, MMP11, PKIB, PRPS1, PSMD10, SH3BP5, SLC16A3, SLC7A11, SPP1, TNNT1, and UBE2E1; and (b) predicting a likelihood of recurrence of BC for the patient based on the expression level of the one or more genes, wherein increased expression of AGR2, CLDN7, EZR, MMP11, PKIB, PRPS1, PSMD10, SLC16A3, SLC7A11, SPP1, TNNT1, and UBE2E1 is correlated with an increased risk of a recurrence of BC, and wherein increased expression of B4GALT1, GNG11, JUN, and SH3BP5 is correlated with a reduced risk of a recurrence of BC. In certain embodiments, the method may additionally comprise normalizing the expression level of the one or more genes selected to obtain a normalized expression level for the one or more genes selected. In further embodiments, the likelihood of recurrence of BC is predicted based on the normalized expression level for the one or more genes selected.

In some embodiments, the BC recurrence is local or locoregional recurrence or distant recurrence (metastasis). In some embodiments, the biological sample is a biopsy or a tumor sample.

As used herein, “risk of BC recurrence” or “likelihood of BC recurrence” refers to a statistical probability (e.g., likelihood) of BC recurrence over an extended period of time (e.g., 1 month, 1 year, 5 years, 10 years, etc.). In some embodiments, risk of BC recurrence involves a baseline wherein a patient has had a successful intervention (e.g., surgical intervention) and is characterized as not having BC and/or actively progressing cancer cells. As such, a risk or likelihood of recurrence involves the likelihood that the cancer will recur in some manner. Such risks can be characterized as very low risk, low risk, moderately low risk, average risk, moderately high risk, high risk, and very high risk.

In some embodiments, increased expression levels of AGR2, CLDN7, EZR, MMP11, PKIB, PRPS1, PSMD10, SLC16A3, SLC7A11, SPP1, TNNT1, and UBE2E1 are each correlated with an increased risk or likelihood of a breast cancer recurrence.

In some embodiments, decreased expression levels of AGR2, CLDN7, EZR, MMP11, PKIB, PRPS1, PSMD10, SLC16A3, SLC7A11, SPP1, TNNT1, and UBE2E1 are each correlated with a decreased risk or likelihood of a breast cancer recurrence.

In some embodiments, increased expression levels of B4GALT1, GNG11, JUN, and SH3BP5 are each correlated with a decreased risk or likelihood of a breast cancer recurrence.

In some embodiments, decreased expression levels of B4GALT1, GNG11, JUN, and SH3BP5 are each correlated with an increased or likelihood risk of a breast cancer recurrence.

In some embodiments, the methods further comprise treating the patient with adjuvant radiotherapy if the patient is characterized as at increased or high risk or likelihood for BC recurrence. In certain embodiments, a patient may be treated adjuvant radiotherapy if the patient is characterized as at moderately high risk, high risk, or very high risk for BC recurrence. In some embodiments, the methods further comprise not treating the patient with adjuvant radiotherapy treatment if the patient is characterized as at a decreased or low risk or likelihood for BC recurrence. In certain embodiments, a patient may not be treated with adjuvant radiotherapy or be identified as a patient who would not benefit from adjuvant radiotherapy if the patient is characterized as at moderately low risk, low risk, or very low risk for BC recurrence.

In some embodiments, the expression levels of all of the genes are measured in the biological sample. In some embodiments, the number of the one or more genes is selected from the group consisting of: 1 gene, 1-2 genes, 1-3, 1-4, 1-5. 1-6, 1-7, 1-8, 1-9, 1-10, 1-11, 1-12, 1-13, 1-14, 1-15, 1-16, 2-3, 2-4, 2-5, 2-6, 2-7, 2-8, 2-9, 2-10, 2-11, 2-12, 2-13, 2-14, 2-15, 2-16, 3-4, 3-5, 3-6, 3-7, 3-8, 3-9, 3-10, 3-11, 3-12, 3-13, 3-14, 3-15, 3-16, 4-5, 4-6, 4-7, 4-8, 4-9, 4-10, 4-11, 4-12, 4-13, 4-14, 4-15, 4-16, 5-6, 5-7, 5-8, 5-9, 5-10, 5-11, 5-12, 5-13, 5-14, 5-15, 5-16, 6-7, 6-8, 6-9, 6-10, 6-11, 6-12, 6-13, 6-14, 6-15, 6-16, 7-8, 7-9, 7-10, 7-11, 7-12, 7-13, 7-14, 7-15, 7-16, 8-9, 8-10, 8-11, 8-12, 8-13, 8-14, 8-15, 8-16, 9-10, 9-11, 9-12, 9-13, 9-14, 9-15, 9-16, 10-11, 10-12, 10-13, 10-14, 10-15, 10-16, 11-12, 11-13, 11-14, 11-15, 11-16, 12-13, 12-14, 12-15, 12-16, 13-14, 13-15, 13-16, 14-15, 14-16, 15-16, and 16 genes.

In some embodiments, measuring the levels of expression comprises performing one or more of: in situ hybridization, a PCR-based method, an array-based method, an immunohistochemical method, an RNA assay method, or an immunoassay method. In some embodiments, measuring the levels of expression comprises using a reagent selected from the group consisting of a nucleic acid probe, one or more nucleic acid primers, and an antibody. In some embodiments, measuring the level of expression comprises measuring the level of an RNA transcript.

In certain embodiments, the disclosure provides a method for prognosing and/or predicting benefit from adjuvant radiotherapy in a subject having BC, the method comprising: a) obtaining or having obtained an expression level in a sample from a subject for one or more genes selected from AGR2, B4GALT1, CLDN7, EZR, GNG11, JUN, MMP11, PKIB, PRPS1, PSMD10, SH3BP5, SLC16A3, SLC7A11, SPP1, TNNT1, and UBE2E1; and b) determining that the subject is at low risk or high risk of cancer recurrence based on the expression level, and/or likely to benefit or not likely benefit from adjuvant radiotherapy based on the expression level, thereby prognosing and/or predicting benefit from adjuvant radiotherapy in the subject. In some embodiments, the method further comprises withholding adjuvant radiotherapy therapy if the subject is identified as not likely to benefit from adjuvant radiotherapy and/or administering a cancer treatment other than adjuvant radiotherapy. In other embodiments, the cancer recurrence is local or locoregional recurrence or distant recurrence (metastasis). In some embodiments, the levels of expression of the one or more of the genes may be increased or decreased compared to a control. In some embodiments, the expression levels of all of the one or more genes are measured in the biological sample. In some embodiments, the method further comprises treating the subject with adjuvant radiotherapy. In some embodiments, the method further comprises treating the subject with a cancer therapy other than adjuvant radiotherapy. In still other embodiments, the method further comprises treating the subject with mastectomy, radiation boost, or adjuvant systemic therapy. In some embodiments, radiotherapy is withheld from the subject following breast conserving surgery (BCS). In some embodiments, the method further comprises determining that the subject is at low risk of cancer recurrence based on the age of the subject, or determining that the subject is not at low risk of cancer recurrence based on the age of the subject. In some embodiments, the cancer recurrence is local or locoregional recurrence or distant recurrence (metastasis). In other embodiments, the subject is at high risk for cancer recurrence and treated with radiotherapy.

In some embodiments, the disclosure provides a method comprising: a) obtaining or having obtained an expression level in a sample from a subject for one or more genes selected from AGR2, B4GALT1, CLDN7, EZR, GNG11, JUN, MMP11, PKIB, PRPS1, PSMD10, SH3BP5, SLC16A3, SLC7A11, SPP1, TNNT1, and UBE2E1; and b) determining that the subject is at low risk of cancer recurrence and not likely to benefit from treatment with adjuvant radiotherapy based on the expression level, or determining that the subject is not at high risk of cancer recurrence and likely to benefit from treatment with adjuvant radiotherapy based on the expression level. In other embodiments, the method further comprises administering a cancer therapy other than adjuvant radiotherapy therapy if the subject is identified as not likely to benefit from adjuvant radiotherapy. In other embodiments, the cancer recurrence is local or locoregional recurrence or distant recurrence (metastasis). In some embodiments, the levels of expression of the one or more of the genes may be increased or decreased compared to a control. In some embodiments, the expression levels of all of the genes selected from Table 5 are measured in the biological sample. In still other embodiments, the method further comprises treating the subject with mastectomy, radiation boost, or adjuvant systemic therapy. In some embodiments, radiotherapy is withheld from the subject following breast conserving surgery (BCS). In some embodiments, the method further comprises determining that the subject is at low risk of cancer recurrence based on the age of the subject, or determining that the subject is not at low risk of cancer recurrence based on the age of the subject. In some embodiments, the cancer recurrence is local or locoregional recurrence or distant recurrence (metastasis).

In some embodiments, the disclosure provides a method of treating breast cancer in a subject, comprising: a) obtaining or having obtained an expression level in a sample from a subject for one or more genes selected from AGR2, B4GALT1, CLDN7, EZR, GNG11, JUN, MMP11, PKIB, PRPS1, PSMD10, SH3BP5, SLC16A3, SLC7A11, SPP1, TNNT1, and UBE2E1; b) determining that the subject is at low risk of cancer recurrence and not likely to benefit from treatment with adjuvant radiotherapy based on the expression level; and c) administering a cancer treatment other than adjuvant radiotherapy therapy if the subject is identified as not likely to benefit from adjuvant radiotherapy based on the expression level. In other embodiments, the cancer recurrence is local or locoregional recurrence or distant recurrence (metastasis). In some embodiments, the levels of expression of one or more of the genes may be increased or decreased compared to a control. In some embodiments, the expression levels of all of the genes are measured in the biological sample. In still other embodiments, the method further comprises treating the subject with mastectomy, radiation boost, or adjuvant systemic therapy. In some embodiments, radiotherapy is withheld from the subject following breast conserving surgery (BCS). In some embodiments, the method further comprises determining that the subject is at low risk of cancer recurrence based on the age of the subject, or determining that the subject is not at low risk of cancer recurrence based on the age of the subject. In some embodiments, the cancer recurrence is local or locoregional recurrence or distant recurrence (metastasis).

In some embodiments, the plurality of genes used in the methods and genomic classifiers of the present disclosure are selected from the group consisting of Anterior Gradient 2, Protein Disulphide Isomerase (AGR2), Beta-1,4-Galactosyltransferase 1 (B4GALT1), Claudin 7 (CLDN7), Ezrin (EZR), G Protein Subunit Gamma 11 (GNG11), Jun Proto Oncogene (JUN), Matrix Metallopeptidase 11 (MMP11), CAMP-Dependent Protein Kinase Inhibitor Beta (PKIB), Phosphoribosyl Pyrophosphate Synthetase 1 (PRPS1), Proteasome 26S Subunit, Non ATPase 10 (PSMD10), SH3 Domain Binding Protein 5 (SH3BP5), Solute Carrier Family 16 Member 3 (SLC16A3), Solute Carrier Family 7 Member 11 (SLC7A11), Secreted Phosphoprotein 1 (SPP1), Troponin T1, Slow Skeletal Type (TNNT1), and Ubiquitin Conjugating Enzyme E2 El (UBE2E1).

In certain embodiments, the subject has estrogen receptor positive (ER+) breast cancer, human epidermal growth factor receptor 2 negative (HER2−) breast cancer, Stage I-II breast cancer, or node-negative breast cancer and/or is post-menopausal.

Such methods are not limited to a specific sample or biological sample type. For example, in some embodiments the sample or biological sample is a tissue sample, bodily fluid sample, blood sample, organ secretion sample, CSF sample, saliva sample, plasma sample, serum sample, or urine sample. In some embodiments, the sample may comprise breast tissue, or surrounding tissue, a breast biopsy, a tumor sample, or tissue that contains breast cells, or breast cancer cells.

In certain embodiments, nucleic acids comprising sequences from genes selected from Table 5, or complements thereof, are isolated from the biological sample, and/or purified, and/or amplified prior to analysis. In some embodiments, the nucleic acids may comprise RNA transcripts.

In other embodiments, the expression levels of biomarkers are determined by in situ hybridization, PCR-based methods, array-based methods, immunohistochemical methods, RNA assay methods, or immunoassay methods. In other embodiments, the levels of gene expression are determined using one or more reagents. In certain embodiments, the one or more reagents are nucleic acid probes, nucleic acid primers, and/or antibodies. In other embodiments, determining the level of expression of a biomarker comprises measuring the level of a nucleic acid. In some embodiments, the nucleic acid is an RNA transcript.

In some embodiments, the level of expression of at least one gene is reduced compared to a control. In other embodiments, the level of expression of at least one gene is increased compared to a control.

In certain embodiments, the methods described herein are performed prior to treatment of the subject with adjuvant radiotherapy. In certain embodiments, the methods described herein are performed prior to treatment of the subject with mastectomy, radiation boost, or adjuvant systemic therapy.

In other embodiments, the method further comprises calculating a risk score for the subject, wherein adjuvant radiotherapy is withheld from the subject if the subject is identified as being at low risk of cancer recurrence and not likely to benefit from adjuvant radiotherapy based on both the risk score and the expression levels of the one or more genes selected from AGR2, B4GALT1, CLDN7, EZR, GNG11, JUN, MMP11, PKIB, PRPS1, PSMD10, SH3BP5, SLC16A3, SLC7A11, SPP1, TNNT1, and UBE2E1 in the biological sample, and administering a cancer therapy other than adjuvant radiotherapy to the subject. In some embodiments, the cancer recurrence is local or locoregional recurrence or distant recurrence (metastasis).

The significance of the expression levels of one or more biomarker genes may be evaluated using, for example, a T-test, P-value, KS (Kolmogorov Smirnov) P-value, accuracy, accuracy P-value, positive predictive value (PPV), negative predictive value (NPV), sensitivity, specificity, AUC, AUC P-value (Auc.pvalue), Wilcoxon Test P-value, Median Fold Difference (MFD), Kaplan Meier (KM) curves, survival AUC (survAUC), Kaplan Meier P-value (KM P-value), Univariable Analysis Odds Ratio P-value (uvaORPval), multivariable analysis Odds Ratio P-value (mvaORPval), Univariable Analysis Hazard Ratio P-value (uvaHRPval) and Multivariable Analysis Hazard Ratio P-value (mvaHRPval). The significance of the expression level of the one or more targets may be based on two or more metrics selected from the group comprising AUC, AUC P-value (Auc.pvalue), Wilcoxon Test P-value, Median Fold Difference (MFD), Kaplan Meier (KM) curves, survival AUC (survAUC), Univariable Analysis Odds Ratio P-value (uvaORPval), multivariable analysis Odds Ratio P-value (mvaORPval), Kaplan Meier P-value (KM P-value), Univariable Analysis Hazard Ratio P-value (uvaHRPval) or Multivariable Analysis Hazard Ratio P-value (mvaHRPval).

In another aspect, the disclosure includes a probe set for determining a prognosis of a subject having BC and whether or not to treat the subject with radiotherapy, the probe set comprising a plurality of probes for detecting a plurality of target nucleic acids, wherein the plurality of target nucleic acids comprises one or more gene sequences, or complements thereof, of one or more genes selected from AGR2, B4GALT1, CLDN7, EZR, GNG11, JUN, MMP11, PKIB, PRPS1, PSMD10, SH3BP5, SLC16A3, SLC7A11, SPP1, TNNT1, and UBE2E1. Probes may be detectably labeled to facilitate detection. In some embodiments, the prognosis comprises cancer recurrence prognosis. In further embodiments, the cancer recurrence is local or locoregional recurrence or distant recurrence (metastasis).

In another aspect, the disclosure includes a system for determining a prognosis of a subject who has BC and whether or not to treat the subject with radiotherapy, the system comprising: a) a probe set described herein; and b) a computer model or algorithm for analyzing an expression level or expression profile of the plurality of target nucleic acids hybridized to the plurality of probes in a biological sample from a subject who has BC and determining if the subject is at low risk of cancer recurrence based on the expression level or expression profile and should be treated with radiotherapy. In some embodiments, the cancer recurrence is local or locoregional recurrence or distant recurrence (metastasis).

In some embodiments, the disclosure includes a kit for determining a prognosis of a subject having breast cancer and whether or not to treat the subject with adjuvant radiotherapy, the kit comprising agents for measuring levels of expression of one or more genes selected from AGR2, B4GALT1, CLDN7, EZR, GNG11, JUN, MMP11, PKIB, PRPS1, PSMD10, SH3BP5, SLC16A3, SLC7A11, SPP1, TNNT1, and UBE2E1. In other embodiments, the kit may include one or more agents (e.g., hybridization probes, PCR primers, or microarray) for measuring levels of expression of a plurality of genes, a container for holding a biological sample comprising breast cancer cells isolated from a human subject for testing, and/or printed instructions for reacting the agents with the biological sample or a portion of the biological sample to determine if the subject is at low risk of cancer recurrence of the breast cancer and likely to benefit from treatment with adjuvant radiotherapy. In some embodiments, the cancer recurrence is local or locoregional recurrence or distant recurrence (metastasis). In other embodiments, the agents are packaged in separate containers. In yet other embodiments, the kit further comprises one or more control reference samples or other reagents for measuring gene expression (e.g., reagents for performing PCR, RT-PCR, microarray analysis, a Northern blot, an immunoassay, or immunohistochemistry). In another embodiment, the kit comprises agents for measuring the levels of expression of all of the following genes: AGR2, B4GALT1, CLDN7, EZR, GNG11, JUN, MMP11, PKIB, PRPS1, PSMD10, SH3BP5, SLC16A3, SLC7A11, SPP1, TNNT1, and UBE2E1. In certain embodiments, the kit comprises a probe set, as described herein, for detecting a plurality of target nucleic acids, wherein the plurality of target nucleic acids comprises one or more gene sequences, or complements thereof, of one or more genes selected from AGR2, B4GALT1, CLDN7, EZR, GNG11, JUN, MMP11, PKIB, PRPS1, PSMD10, SH3BP5, SLC16A3, SLC7A11, SPP1, TNNT1, and UBE2E1, or any combination thereof.

In other embodiments, the kit further comprises a system, wherein the system comprises: a) a probe set comprising a plurality of probes for detecting a plurality of target nucleic acids, wherein the plurality of target nucleic acids comprises one or more gene sequences, or complements thereof, of one or more genes selected from AGR2, B4GALT1, CLDN7, EZR, GNG11, JUN, MMP11, PKIB, PRPS1, PSMD10, SH3BP5, SLC16A3, SLC7A11, SPP1, TNNT1, and UBE2E1; and b) a computer model or algorithm for analyzing an expression level or expression profile of the plurality of target nucleic acids hybridized to the plurality of probes in a biological sample from a subject who has breast cancer and determining if the subject is at low risk of cancer recurrence based on the expression level or expression profile and not likely to benefit from treatment with adjuvant radiotherapy. In some embodiments, the cancer recurrence is local or locoregional recurrence or distant recurrence (metastasis).

These and other embodiments of the subject disclosure will readily occur to those of skill in the art in view of the disclosure herein.

### BRIEF DESCRIPTION OF THE DRAWINGS

FIG. 1: Diagram for selection of training and validation cohorts in SweBCG91-RT.

FIG. 2: Diagram for selection of Princess Margaret validation cohort.

FIG. 3: Diagram for selection of genes to be included in the model.

FIG. 4: Cumulative incidence of locoregional recurrence with or without adjuvant radiotherapy (RT) in the SweBCG91-RT validation cohort for patients classified by POLAR as low risk (A) or high risk (B). Hazard ratios and p-values are calculated using a cause-specific Cox proportional hazards regression model.

FIG. 5: Cumulative incidence of locoregional recurrence in the Princess Margaret cohort with or without adjuvant radiotherapy (RT) for patients classified by POLAR as low risk (A) or high risk (B). Hazard ratios and p-values are calculated using a cause-specific Cox proportional hazards regression model.

### DETAILED DESCRIPTION OF THE INVENTION

Multiple phase III randomized clinical trials have consistently demonstrated the benefit of whole-breast radiotherapy (RT) after breast conserving surgery (BCS) in reducing locoregional recurrence (LRR), and RT is considered the standard of care for women with early-stage invasive breast cancer (see, Malmström, P. et al. European journal of cancer (Oxford, England: 1990) 39, 1690-1697 (2003); Fyles, A. W. et al. The New England journal of medicine 351, 963-970 (2004); Holli, K., et al., Br J Cancer 84, 164-169 (2001); Forrest, A. P. et al. Lancet 348, 708-713 (1996); Fisher, B. et al. Journal of clinical oncology: official journal of the American Society of Clinical Oncology 20, 4141-4149 (2002); Liljegren, G. et al. J Clin Oncol 17, 2326-2333 (1999); Veronesi, U. et al. Annals of oncology: official journal of the European Society for Medical Oncology/ESMO 12, 997-100 (2001); Clark, R. M. et al. J Natl Cancer Inst 88, 1659-1664 (1996)). The Oxford Early Breast Cancer Trialists' Group (EBCTCG) meta-analysis demonstrated a two-thirds relative risk reduction of local recurrence at 10-years for patients who receive RT, from 30% to 10%, which translated into a survival benefit of ˜5% at 15 years (see, Clarke, M. et al. Lancet 366, 2087-2106 (2005); Early Breast Cancer Trialists' Collaborative, Lancet 378, 1707-1716 (2011)). In addition to the improvement in locoregional disease control, these data also demonstrated the heterogeneity in response and benefit from adjuvant radiation. Even in the previous era of less effective systemic chemotherapy and limited use of endocrine therapy, up to of 70% of women did not recur locally without RT, yet clinical tools to identify which women may safely forgo adjuvant radiation therapy are still lacking.

Clinical risk factors such as older age, smaller tumor size, and estrogen receptor positive (ER+) breast cancers have been associated with low risk of LRR (see, Fredriksson, I. et al. The British journal of surgery 90, 1093-1102 (2003)). Attempts to use these features to identify women who may omit radiotherapy after BCS have had mixed outcomes. The CALGB 9343 trial enrolled women 70 years or older with small, ER+ breast cancers resected with negative margins who were then randomized to receive whole breast RT and tamoxifen for 5 years or only tamoxifen (see, Hughes, K. S. et al. Journal of clinical oncology: official journal of the American Society of Clinical Oncology 31, 2382-2387 (2013)). In this trial of over 600 women, RT decreased the risk of LRR at 10-years from 10% to 2% without impacting the rate of breast preservation. Similarly, the PRIME II trial included patients aged 65 years or older with small ER+breast cancers resected with negative margins who were treated with endocrine therapy and randomized to RT or not. The recently reported 10-year results demonstrated a decrease in IBTR from 9.8% to 0.9% with the addition of RT (see, Kunkler, I. H. et al. The lancet oncology 16, 266-273 (2015)). As the rate of local recurrence at 10 years for those not treated with RT was 10% in both trials, it can be argued that the 80-90% relative risk reduction at 10 years with the addition of radiation therapy may be too significant to omit RT in women who have a median life expectancy of above 80 years in the United States. Thus far no subtype or clinicopathologic variable has been incorporated in clinical practice that predicts lack of benefit of adjuvant breast radiation, making radiation omission decisions challenging clinically (see, Killander, F. et al. European journal of cancer (Oxford, England: 1990) 67, 57-65 (2016); Sjöström, M. et al. Journal of Clinical Oncology 35, 3222-3229 (2017); Liu, F. F. et al. Journal of clinical oncology: official journal of the American Society of Clinical Oncology 33, 2035-2040 (2015)).

Interest in the use of genomically-informed risk stratifiers has grown in recent years given the success of such approaches in determining likelihood of systemic chemotherapy benefit in women with breast cancer. Such molecularly informed tests have been shown to be prognostic of outcome in women with breast cancer and/or predictive of benefit of chemotherapy in previous clinical trials (see, Albain, K. S. et al. The Lancet Oncology 11, 55-65, (2010); Cardoso, F. et al. New England Journal of Medicine 375, 717-729, (2016): Dowsett, M. et al. Journal of Clinical Oncology 28, 1829-1834 (2010); Dowsett, M. et al. Journal of Clinical Oncology 31, 2783-2790 (2013); Paik, S. et al. The New England journal of medicine 351, 2817-2826, (2004); Sparano, J. A. et al. New England Journal of Medicine 373, 2005-2014 (2015)). While previous attempts to build local recurrence or radiation sensitivity signatures have been made, there exist no widely used signatures to select for patients who may omit radiation in early stage invasive breast cancer (see, Cui, Y., Li, B., Pollom, E. L., Horst, K. C. & Li, R. Clinical cancer research: an official journal of the American Association for Cancer Research 24, 4754-4762 (2018); Eschrich, S. A. et al. International journal of radiation oncology, biology, physics 75, 489-496 (2009); Sjöström, M. et al. Journal of Clinical Oncology 37, 3340-3349, (2019); Speers, C. et al. Clinical cancer research: an official journal of the American Association for Cancer Research 21, 3667-3677 (2015); Tramm, T. et al. Clinical cancer research: an official journal of the American Association for Cancer Research 20, 5272-5280 (2014)). ARTIC, a 27-gene clinicogenomic signature, was previously developed and validated as prognostic for LRR and predictive for benefit from RT. This signature was able to identify patients at higher risk of LRR, with a reduced benefit from radiotherapy, and thus may be used to identify those who require intensified treatment. Patients identified as low risk by ARTIC had greater benefit from RT and were not suitable candidates for RT omission. Signatures designed to estimate risk of distant recurrence after systemic therapy have been suggested for use for avoidance of RT, and these are being tested in the context of ongoing clinical trials, but these trials will not mature for several years (see, Speers, C. & Pierce, L. J. Current Breast Cancer Reports 12, 255-265 (2020)).

Experiments conducted during the course of developing embodiments for the present invention (see, Example I) performed an analysis of SweBCG91-RT, a trial randomizing women with node-negative stage I-II invasive breast cancer (BC)+/−RT following BCS, and the Princess Margaret cohort which randomized women age 50 years or older with T1-T2 node-negative BC to +/−RT following BCS. Patients in the SweBCG91-RT trial were not treated with adjuvant systemic therapy, while patients in the Princess Margaret trial were treated with tamoxifen. Transcriptome-wide profiling of tumors was performed using the Affymetrix Human Exon 1.0 ST microarray. The SweBCG91-RT cohort was divided into a training cohort of 243 patients and a validation cohort of 354 patients, and a 16-gene signature (AGR2, B4GALT1, CLDN7, EZR, GNG11, JUN, MMP11, PKIB, PRPS1, PSMD10, SH3BP5, SLC16A3, SLC7A11, SPP1, TNNT1, and UBE2E1; see Table 5 and Example I) was trained to predict LRR using elastic net regression, named Profile for the Omission of Local Adjuvant Radiation (POLAR). It was shown that patients categorized as POLAR low-risk and not treated with RT had a 10-year LRR of 6%, that there was no significant benefit from RT in POLAR low-risk patients, and patients categorized as POLAR high-risk had a significant decreased risk of LRR when treated with RT. These results indicate that the novel POLAR genomic signature based on LRR biology is capable of identifying patients with a low risk of LRR despite not receiving RT, and thus prime candidates for RT omission.

Accordingly, the present disclosure relates to systems and methods for providing individualized prognostic assessments of BC recurrence (e.g., LRR). The systems and methods involve measuring gene expression from a patient sample to create a gene expression signature which identifies subjects who are not likely to benefit from RT.

In certain embodiments, the present invention provides methods, comprising: a) measuring an expression level of one or more genes in a biological sample from a human patient having or at risk of having breast cancer (BC), wherein the one or more genes are selected from AGR2, B4GALT1, CLDN7, EZR, GNG11, JUN, MMP11, PKIB, PRPS1, PSMD10, SH3BP5, SLC16A3, SLC7A11, SPP1, TNNT1, and UBE2E1; and b) determining a likelihood of BC recurrence for the patient based on the expression level of the one or more genes.

In certain embodiments, the disclosure provides methods for predicting a likelihood of recurrence of BC for a patient with BC at risk for having BC comprising: (a) measuring, in a sample obtained from the patient, an expression level of one or more of the following genes: AGR2, B4GALT1, CLDN7, EZR, GNG11, JUN, MMP11, PKIB, PRPS1, PSMD10, SH3BP5, SLC16A3, SLC7A11, SPP1, TNNT1, and UBE2E1; and (b) predicting a likelihood of recurrence of BC for the patient based on the expression level of the one or more genes, wherein increased expression of AGR2, CLDN7, EZR, MMP11, PKIB, PRPS1, PSMD10, SLC16A3, SLC7A11, SPP1, TNNT1, and UBE2E1 is correlated with an increased risk of a recurrence of BC, and wherein increased expression of B4GALT1, GNG11, JUN, and SH3BP5 is correlated with a reduced risk of a recurrence of breast cancer.

In certain embodiments, the present disclosure discloses systems and methods for diagnosing, predicting, and/or monitoring the status or outcome of a BC in a subject using expression-based analysis of one or more genes/gene targets. Generally, the method comprises (a) obtaining an expression level in a sample from a subject for one or more genes; and (b) determining that the subject's risk of cancer recurrence based on the expression level of the one or more genes. In some embodiments, the method may also comprise either administering RT if the subject is identified as being at risk of cancer recurrence based on the expression level of the one or more genes, or withholding RT if the subject is identified as being at low risk of cancer recurrence based on the expression level of the one or more genes.

In certain embodiments, methods for determining if a subject is at low or high risk of recurrence of the breast cancer are provided. Generally, the method comprises: (a) providing a sample comprising breast cancer cells from a subject; (b) assaying the expression level for one or more genes in the sample; and (c) determining if the subject is at low risk of recurrence of the breast cancer based on the expression level of the plurality of targets. In certain embodiments, the method may additionally comprise determining whether or not to treat the subject with adjuvant radiotherapy, chemotherapy, or endocrine therapy. For example, a subject identified as being at low risk of recurrence of the breast cancer according to the methods of the present disclosure, may be less likely or not likely to respond to adjuvant radiotherapy, whereas a subject identified as being at higher risk of recurrence of the breast cancer may be more likely to respond to adjuvant radiotherapy.

In certain embodiments, this disclosure relates to systems and methods for providing individualized prognostic assessments of BC recurrence, and the identification of subjects who are not likely to benefit from RT. Such systems and methods of the present invention are not limited to a particular manner of assessing risk of BC recurrence. In some embodiments, the risk of BC recurrence assessment is provided in the format of gene expression signatures that give important, and easy to understand, information about patient tumors.

In certain embodiments, systems and methods of the present invention involve measuring and analyzing the gene expression from a patient sample to create a gene expression signature. In some embodiments, the gene expression signature is provided as an easy-to-understand score or risk category. For example, the gene expression signature may be provided as a numerical risk score, or the risk score may be used to assign the patient to a category for risk of BC recurrence. For instance, a patient may be categorized as high or low risk for BC recurrence. In other embodiments, risk category may be characterized as very low risk, low risk, moderately low risk, average or intermediate risk, moderately high risk, high risk, or very high risk of BC recurrence. The risk score or risk category may make talking to patients about their test results easy and efficient and may also help the physician make treatment decisions more quickly by reducing the amount of time required to interpret patient results.

Therefore, methods of the invention involve, in certain embodiments, the creation of risk scores useful for the clinical management of breast cancer. The gene expression signatures may predict, for example, a risk of disease recurrence, and as such, the gene expression signatures may be used to select an optimal course of treatment. For example, the risk scores may be used to identify patients that are at a high risk for recurrence and thus good candidates for RT. Alternatively, the risk scores may be used to identify patients that are at a low risk for recurrence and thus good candidates for omitting RT. Accordingly, risk scores may be useful for classifying a patient and selecting an appropriate treatment.

For instance, in some embodiments, expression measurements for selected genes or combinations thereof may be formulated into linear or non-linear models or algorithms and converted into a likelihood or risk score.

In certain embodiments, the likelihood or risk score may be calculated using a linear algorithm where each gene assayed is assigned a weight or coefficient based on the gene's individual correlation to BC recurrence (see Table 8). The weighted expression levels for the one or more genes assayed may then be added together to produce a likelihood or risk score. In one embodiment, if a single gene's expression level is assayed, the weighted expression level for that gene may be considered the risk score and may be used to determine the patient's risk category.

In certain embodiments, the resulting score may comprise a numerical value, and may be in a range between −5.0 and 10.0, or between −4.0 and 9.0, or between −3.0 and 8.0, or between −2.0 and 7.0, or between −1.0 and 6.0, or between −0.5 and 5.0, or between −0.5 and 4.0, or between −0.5 and 3.0, or between 0 and 2.5, or between 0.5 and 2.0, or between 1.0 and 1.5. In further embodiments, the score may comprise for instance, a numerical value such as −5.0, −4.5, −4.0, −3.5, −3.0, −2.5, −2.0, −1.5, −1.0, −0.95, −0.90, −0.85, −0.80, −0.75, −0.70, −0.65, −0.60, −0.55, −0.50, −0.45, −0.40, −0.35, −0.30, −0.25, −0.20, −0.15, −0.10, −0.05, 0, 0.05, 0.10, 0.15, 0.20, 0.25, 0.30, 0.35, 0.40, 0.45, 0.50, 0.55, 0.60, 0.65, 0.70, 0.75. 0.80, 0.85, 0.90, 0.95, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, or 10.0.

In other embodiments, the risk score may be compared to a pre-determined threshold to determine if a patient is at high risk (i.e. with a score above the pre-determined threshold) or at low risk (i.e. with a score below the pre-determined threshold) for BC recurrence and thus likely or not to benefit from RT. In certain embodiments, the pre-determined threshold may be 0, such that a patient with a positive risk score is considered high risk and a patient with a negative risk score is considered low risk. In another embodiment, the pre-determined threshold may fall within a range between −1.0 and 5.0, for instance between −0.5 and 3.0, between 0 and 2.0, between 0 and 1.0, between 0.25 and 0.75, between 0.30 and 0.70, between 0.40 and 0.60, between 0.50 and 0.60, between 0.55 and 0.65, between 0.55 and 0.60, and between 0.60 and 0.65. In a further embodiment, the pre-determined threshold may include −1.0, −0.90, −0.80, −0.70, −0.60, −0.55, −0.50, −0.45, −0.40, −0.35, −0.30, −0.25, −0.20, −0.15, −0.10, −0.05, 0, 0.05, 0.10, 0.15, 0.20, 0,25, 0.30, 0.35, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.75, 0.80, 0.85, 0.90, 0.95, 1.0, 1.5, 2.0, 2.5, or 3.0.

In further embodiments, a risk or likelihood of recurrence may be characterized as very low risk, low risk, moderately low risk, average or intermediate risk, moderately high risk, high risk, or very high risk. In such embodiments, the risk score may be compared to more than one pre-determined thresholds to determine if a patient is at very low risk, low risk, moderately low risk, average or intermediate risk, moderately high risk, high risk, or very high risk of BC recurrence or any subset or combination of these risk levels. For instance, the risk score may be compared to more than one pre-determined thresholds to determine if a patient is at low risk, average or intermediate risk, or high risk of BC recurrence. In another embodiment, the risk score may be compared to more than one pre-determined thresholds to determine if a patient is at very low risk, low risk, average or intermediate risk, high risk, or very high risk of BC recurrence. In such cases where the risk score is to be compared to more than one pre-determined thresholds, the pre-determined thresholds may fall within a range between −1.0 and 5.0, for instance between −0.5 and 3.0, between 0 and 2.0, between 0 and 1.0, between 0.25 and 0.75, between 0.30 and 0.70, between 0.40 and 0.60, between 0.50 and 0.60, between 0.55 and 0.65, between 0.55 and 0.60, and between 0.60 and 0.65. In a further embodiment, the pre-determined thresholds may include −1.0, −0.90. −0.80, −0.70, −0.60. −0.55, −0.50, −0.45. −0.40, −0.35, −0.30, −0.25, −0.20, −0.15, −0.10, −0.05, 0, 0.05, 0.10, 0.15, 0.20, 0.25, 0.30, 0.35, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57. 0.58, 0.59, 0.60, 0.61, 0.62, 0.63, 0.64, 0.65, 0.66, 0.67, 0.68, 0.69, 0.70, 0.75, 0.80, 0.85, 0.90, 0.95, 1.0, 1.5, 2.0, 2.5, or 3.0 or any combination thereof. In certain embodiments, each of the more than one pre-determined thresholds or range of thresholds, may be distinct and may associated with a distinct risk level.

In some embodiments, the risk or likelihood score may be scaled or adjusted to an easily interpreted scale. For instance, the risk score may be scaled or adjusted to a value between 1 and 100. Such easily interpretable scale may make treatments decision and talking to patients about their test results and treatment options easy and efficient.

In certain embodiments, the pre-determined thresholds for determining risk levels may be similarly scaled or adjusted, for instance such that they match or correspond to the adjustment made to obtain the final scale. For example, if the risk score is scaled or adjusted to a scale with values between 1 and 100, the pre-determined threshold, or more than one pre-determined thresholds, may be scaled or adjusted accordingly such that the values of such thresholds fall within the 1-100 scale.

Methods of the invention may further include combining expression signatures or risk scores with other clinical factors to give a single risk appraisal.

As used herein, “risk of BC recurrence” or “likelihood of BC recurrence” refers to a statistical probability (e.g., likelihood) of BC recurrence over an extended period of time (e.g., 1 month, 1 year, 5 years, 10 years, etc.). In some embodiments, risk of BC recurrence involves a baseline wherein a patient has had a successful intervention (e.g., surgical intervention) and is characterized as not having BC and/or actively progressing cancer cells. As such, a risk or likelihood of recurrence involves the likelihood that the cancer will recur in some manner.

In some embodiments, increased expression levels of AGR2, CLDN7, EZR, MMP11, PKIB, PRPS1, PSMD10, SLC16A3, SLC7A11, SPP1, TNNT1, and UBE2E1 are each correlated with an increased risk or likelihood of a breast cancer recurrence.

In some embodiments, decreased expression levels of AGR2, CLDN7, EZR, MMP11, PKIB, PRPS1, PSMD10, SLC16A3, SLC7A11, SPP1, TNNT1, and UBE2E1 are each correlated with a decreased risk or likelihood of a breast cancer recurrence.

In some embodiments, increased expression levels of B4GALT1, GNG11, JUN, and SH3BP5 are each correlated with a decreased risk or likelihood of a breast cancer recurrence.

In some embodiments, decreased expression levels of B4GALT1, GNG11, JUN, and SH3BP5 are each correlated with an increased or likelihood risk of a breast cancer recurrence.

Such systems and methods are not limited to use of a particular type or kind of sample from a subject. For example, in some embodiments the sample or biological sample is a tissue sample, bodily fluid sample, blood sample, organ secretion sample, CSF sample, saliva sample, plasma sample, serum sample, or urine sample. In some embodiments, the sample may comprise breast tissue, or surrounding tissue, a breast biopsy, a tumor sample, or tissue that contains breast cells, or breast cancer cells.

In some embodiments, the subject is a human subject. In some embodiments, the subject is a human subject at risk for developing BC. In some embodiments, the subject is a female human subject at risk for developing BC. In some embodiments, the subject is a human subject diagnosed with BC. In some embodiments, the subject is a female human subject diagnosed with BC. The subject may be suspected of having a cancer on account of various symptoms including the detection of a lump or mass. In some embodiments, the cancer is early stage breast cancer, i.e., cancer that is contained entirely within the breast.

In some embodiments, gene expression analysis or comparison may be performed with an unsupervised, hierarchical clustering algorithm, such as a K-means clustering algorithm. A clustering algorithm is an algorithm that clusters or groups a set of objects in such a way that the objects in the same group (called a cluster) are more like each other than to those in other groups (clusters). The clustering algorithm may cluster RNA expression levels from the patient sample with the RNA expression levels expected in one or more stages of cancer. The RNA expression levels expected in patients having a low risk of BC recurrence or a high risk of BC recurrence may come from one or more tumor samples associated with known outcomes. The RNA expression levels may be clustered based on their similarities of expression. In some embodiments, the clustering algorithm clusters the RNA expression levels into distinct groups associated with the known outcomes. The groups may reflect a continuum of outcomes that are indicative of prognoses.

Such systems and methods are not limited to a particular manner or technique for measuring and/or determining gene expression. “Gene expression” as used herein refers to the relative levels of expression and/or pattern of expression of a gene in a biological sample.

Techniques for measuring gene expression are known in the art. Indeed, any known technique for measuring gene expression is contemplated and herein incorporated. Gene expression can be determined by any suitable technique including, but not limited to techniques comprising PCR based techniques (e.g., real-time PCR), gel electrophoresis techniques, chromatographic techniques, antibody-based techniques, centrifugation techniques, or combinations thereof. Methods for measuring gene expression can comprise measuring amounts of cDNA made from tissue-isolated RNA. In some embodiments, gene expression measurement techniques involve gene expression assays with or without the use of gene chips (see, Onken et al., J Molec Diag 12(4): 461-468 (2010); and Kirby et al., Adv Clin Chem 44: 247-292 (2007). In some embodiments, gene expression measurement techniques involve affymetrix gene chips and RNA chips and gene expression assay kits (e.g., Applied Biosystems™ TaqMant Gene Expression Assays).

Additional techniques for determining the level of gene expression in a biological sample involves the process of nucleic acid amplification, for example, by RT-PCR (U.S. Pat. No. 4,683,202), ligase chain reaction (Barany, Proc. Natl. Acad. Sci. USA 88:189-93, 1991), self sustained sequence replication (Guatelli et al., Proc. Natl. Acad. Sci. USA 87:1874-78, 1990), transcriptional amplification system (Kwoh et al., Proc. Natl. Acad. Sci. USA 86:1173-77, 1989), Q-Beta Replicase (Lizardi et al., Bio/Technology 6:1197, 1988), rolling circle replication (U.S. Pat. No. 5,854,033), or any other nucleic acid amplification method, followed by the detection of the amplified molecules using techniques well known to those of skill in the art.

In some embodiments, gene expression is determined with a quantitative allele-specific real-time target and signal amplification (QuARTS) assay. Three reactions sequentially occur in each QUARTS assay, including amplification (reaction 1) and target probe cleavage (reaction 2) in the primary reaction; and FRET cleavage and fluorescent signal generation (reaction 3) in the secondary reaction. When target nucleic acid is amplified with specific primers, a specific detection probe with a flap sequence loosely binds to the amplicon. The presence of the specific invasive oligonucleotide at the target binding site causes a 5′ nuclease, e.g., a FEN-1 endonuclease, to release the flap sequence by cutting between the detection probe and the flap sequence. The flap sequence is complementary to a non-hairpin portion of a corresponding FRET cassette. Accordingly, the flap sequence functions as an invasive oligonucleotide on the FRET cassette and effects a cleavage between the FRET cassette fluorophore and a quencher, which produces a fluorescent signal. The cleavage reaction can cut multiple probes per target and thus release multiple fluorophores per flap, providing exponential signal amplification. QUARTS can detect multiple targets in a single reaction well by using FRET cassettes with different dyes. See, e.g., in Zou et al. (2010) “Sensitive quantification of methylated markers with a novel methylation specific technology” Clin Chem 56: A199), and U.S. Pat. Nos. 8,361,720; 8,715,937; 8,916,344; and 9,212,392, each of which is incorporated herein by reference for all purposes.

In certain embodiments, gene expression levels may be normalized to minimize errors or variation between samples. Normalization is thus useful make accurate comparisons of gene expression between samples, as gene expression on a per sample basis can be affected by non-biological variables that arise during sample collection and processing, which may add noise to the true signal. Assays can provide for normalization by incorporating the expression of certain normalizing genes, which do not significantly differ in expression levels under the relevant conditions. Exemplary normalization genes disclosed herein include housekeeping genes. (See, e.g., E. Eisenberg, et al., Trends in Genetics 19(7):362-365 (2003).) Normalization can be based on the mean or median signal (Ct or Cp) of all of the assayed genes or a large subset thereof (global normalization approach). In general, the normalizing genes, also referred to as reference genes should be genes that are known not to exhibit significantly different expression in BC as compared to non-cancerous breast tissue, and are not significantly affected by various sample and process conditions, thus provide for normalizing away extraneous effects. For example, reference genes useful in the methods disclosed herein may include genes frequently used in the art to normalize patterns of gene expression, including but not limited to, glyceraldehyde-3-phosphate-dehydrogenase (GAPDH) and β-actin, or any other reference gene known in the art. Those skilled in the art will recognize that normalization may be achieved in numerous ways, and the techniques described above are intended only to be exemplary, not exhaustive.

In some embodiments, assaying the expression level for one or more gene targets in the sample may comprise applying the sample to a microarray.

In certain embodiments, gene expression assayed by microarray may be normalized using the Single Channel Array Normalization (SCAN) method. The SCAN method utilizes a single-sample technique, rather than processing microarray samples as groups, thereby avoiding the biases that can be introduced group processing. This method normalizes each sample individually by modeling the effects of probe-nucleotide composition on fluorescence intensity and removing the probe- and array-specific background noise. (Piccolo S R, Sun Y, Campbell J D, Lenburg M E, Bild A H, Johnson W E (2012). “A single-sample microarray normalization method to facilitate personalized-medicine workflows.” Genomics, 100(6), 337-344).

In some instances, assaying the expression level for one or more genes comprises the use of an algorithm. The algorithm may be used to produce a genomic classifier. Alternatively, the classifier may comprise a probe selection region. In some instances, assaying the expression level for a plurality of targets comprises detecting and/or quantifying the one or more genes. In some embodiments, assaying the expression level for one or more genes comprises sequencing the plurality of targets. In some embodiments, assaying the expression level for one or more gene targets comprises amplifying the plurality of targets. In some embodiments, assaying the expression level for one or more gene targets comprises quantifying the targets. In some embodiments, assaying the expression level for one or more targets comprises conducting a multiplexed reaction on the plurality of targets.

In some instances, assaying the expression level of a plurality of genes comprises detecting and/or quantifying a plurality of target analytes. In some embodiments, assaying the expression level of a plurality of genes comprises sequencing a plurality of target nucleic acids. In some embodiments, assaying the expression level of a plurality of biomarker genes comprises amplifying a plurality of target nucleic acids. In some embodiments, assaying the expression level of a plurality of biomarker genes comprises conducting a multiplexed reaction on a plurality of target analytes.

The methods disclosed herein often comprise assaying the expression level of a plurality of targets. The plurality of targets may comprise coding targets and/or non-coding targets of a protein-coding gene or a non-protein-coding gene. A protein-coding gene structure may comprise an exon and an intron. The exon may further comprise a coding sequence (CDS) and an untranslated region (UTR). The protein-coding gene may be transcribed to produce a pre-mRNA and the pre-mRNA may be processed to produce a mature mRNA. The mature mRNA may be translated to produce a protein.

A non-protein-coding gene structure may comprise an exon and intron. Usually, the exon region of a non-protein-coding gene primarily contains a UTR. The non-protein-coding gene may be transcribed to produce a pre-mRNA and the pre-mRNA may be processed to produce a non-coding RNA (ncRNA).

A coding target may comprise a coding sequence of an exon. A non-coding target may comprise a UTR sequence of an exon, intron sequence, intergenic sequence, promoter sequence, non-coding transcript, CDS antisense, intronic antisense, UTR antisense, or non-coding transcript antisense. A non-coding transcript may comprise a non-coding RNA (ncRNA).

Such systems and methods are not limited to analysis of specific genes. In some embodiments, the one or more genes is selected from AGR2, B4GALT1, CLDN7, EZR, GNG11, JUN, MMP11, PKIB, PRPS1, PSMD10, SH3BP5, SLC16A3, SLC7A11, SPP1, TNNTI, and UBE2E1. Such systems and embodiments are not limited to use of a specific number or combination of the one or more genes (e.g., a combination of only 1 gene, 1-2 genes, 1-3, 1-4, 1-5, 1-6, 1-7, 1-8, 1-9, 1-10, 1-11, 1-12, 1-13, 1-14, 1-15, 1-16, 2-3, 2-4, 2-5, 2-6, 2-7, 2-8, 2-9, 2-10, 2-11, 2-12, 2-13, 2-14, 2-15, 2-16, 3-4, 3-5, 3-6, 3-7, 3-8, 3-9, 3-10, 3-11, 3-12, 3-13, 3-14, 3-15, 3-16, 4-5, 4-6, 4-7, 4-8, 4-9, 4-10, 4-11, 4-12, 4-13, 4-14, 4-15, 4-16, 5-6, 5-7, 5-8, 5-9, 5-10, 5-11, 5-12, 5-13, 5-14, 5-15, 5-16, 6-7, 6-8, 6-9, 6-10, 6-11, 6-12, 6-13, 6-14, 6-15, 6-16, 7-8, 7-9, 7-10, 7-11, 7-12, 7-13, 7-14, 7-15, 7-16, 8-9, 8-10, 8-11, 8-12, 8-13, 8-14, 8-15, 8-16, 9-10, 9-11, 9-12, 9-13, 9-14, 9-15, 9-16, 10-11, 10-12, 10-13, 10-14, 10-15, 10-16, 11-12, 11-13, 11-14, 11-15, 11-16, 12-13, 12-14, 12-15, 12-16, 13-14, 13-15, 13-16, 14-15, 14-16, 15-16, or 16 genes). In some embodiments, the method comprises combinations of 16 or less, 15 or less, 14 or less, 13 or less, 12 or less, 11 or less, 10 or less, 9 or less, 8 or less, 7 or less, 6 or less, 5 or less, 4 or less, 3 or less, or 2 or less genes. In other embodiments, the method may comprise 2 or more, 3 or more, 4 or more, 5 or more, 6 or more, 7 or more, 8 or more, 9 or more, 10 or more, 11 or more, 12 or more, 13 or more, 14 or more, or 15 or more genes. In another embodiment, the one or more genes of the disclosed methods consists of 16 genes.

In some instances, the one or more gene targets comprises a coding target, non-coding target, or any combination thereof. In some instances, the coding target comprises an exonic sequence. In other instances, the non-coding target comprises a non-exonic or exonic sequence. Alternatively, a non-coding target comprises a UTR sequence, an intronic sequence, antisense, or a non-coding RNA transcript. In some instances, a non-coding target comprises sequences which partially overlap with a UTR sequence or an intronic sequence. A non-coding target also includes non-exonic and/or exonic transcripts. Exonic sequences may comprise regions on a protein-coding gene, such as an exon, UTR, or a portion thereof. Non-exonic sequences may comprise regions on a protein-coding, non-protein-coding gene, or a portion thereof. For example, non-exonic sequences may comprise intronic regions, promoter regions, intergenic regions, a non-coding transcript, an exon anti-sense region, an intronic anti-sense region, UTR anti-sense region, non-coding transcript anti-sense region, or a portion thereof. In other instances, the plurality of targets comprises a non-coding RNA transcript.

The gene targets may comprise one or more targets selected from a classifier disclosed herein. The classifier may be generated from one or more models or algorithms. The one or more models or algorithms may be Naïve Bayes (NB), recursive Partitioning (Rpart), random forest (RF), support vector machine (SVM), k-nearest neighbor (KNN), high dimensional discriminate analysis (HDDA), linear model, or a combination thereof. The classifier may have an AUC of equal to or greater than 0.60. The classifier may have an AUC of equal to or greater than 0.61. The classifier may have an AUC of equal to or greater than 0.62. The classifier may have an AUC of equal to or greater than 0.63. The classifier may have an AUC of equal to or greater than 0.64. The classifier may have an AUC of equal to or greater than 0.65. The classifier may have an AUC of equal to or greater than 0.66. The classifier may have an AUC of equal to or greater than 0.67. The classifier may have an AUC of equal to or greater than 0.68. The classifier may have an AUC of equal to or greater than 0.69. The classifier may have an AUC of equal to or greater than 0.70. The classifier may have an AUC of equal to or greater than 0.75. The classifier may have an AUC of equal to or greater than 0.77. The classifier may have an AUC of equal to or greater than 0.78. The classifier may have an AUC of equal to or greater than 0.79. The classifier may have an AUC of equal to or greater than 0.80. The AUC may be clinically significant based on its 95% confidence interval (CI). The accuracy of the classifier may be at least about 70%. The accuracy of the classifier may be at least about 73%. The accuracy of the classifier may be at least about 75%. The accuracy of the classifier may be at least about 77%. The accuracy of the classifier may be at least about 80%. The accuracy of the classifier may be at least about 83%. The accuracy of the classifier may be at least about 84%. The accuracy of the classifier may be at least about 86%. The accuracy of the classifier may be at least about 88%. The accuracy of the classifier may be at least about 90%. The p-value of the classifier may be less than or equal to 0.05. The p-value of the classifier may be less than or equal to 0.04. The p-value of the classifier may be less than or equal to 0.03. The p-value of the classifier may be less than or equal to 0.02. The p-value of the classifier may be less than or equal to 0.01. The p-value of the classifier may be less than or equal to 0.008. The p-value of the classifier may be less than or equal to 0.006. The p-value of the classifier may be less than or equal to 0.004. The p-value of the classifier may be less than or equal to 0.002. The p-value of the classifier may be less than or equal to 0.001.

The one or more gene targets may comprise one or more targets selected from a linear model classifier. The plurality of targets may comprise two or more targets selected from a linear model classifier. The plurality of targets may comprise three or more targets selected from a linear model classifier. The plurality of targets may comprise 2, 3, 4, 5, 6, 7, 8, 9, 10, 11, 12, 13, 14, 15, 20, 25, 27 or more targets selected from a linear model classifier. The linear model classifier may be an LM2, and LM3, or an LM4 classifier. The linear model classifier may be an LM16 classifier (e.g., a linear model classifier with 16 targets). For example, a linear model classifier of the present disclosure may comprise two or more targets selected from Table 5.

The one or more gene targets may comprise one or more targets selected from an SVM classifier. The plurality of targets may comprise 2, 3, 4, 5, 6, 7, 8, 9, 10 or more targets selected from an SVM classifier. The plurality of targets may comprise 12, 13, 14, 15, 17, 20, 22, 25 or more targets selected from an SVM classifier. The SVM classifier may be an SVM2 classifier. An SVM classifier of the present disclosure may comprise two or more targets selected from Table 5.

The one or more gene targets may comprise one or more targets selected from a KNN classifier. The plurality of targets may comprise 2, 3, 4, 5, 6, 7, 8, 9, 10 or more targets selected from a KNN classifier. The plurality of targets may comprise 12, 13, 14, 15, 17, 20, 22, 25 or more targets selected from a KNN classifier. For example, a KNN classifier of the present disclosure may comprise two or more targets selected from Table 5.

The one or more gene targets may comprise one or more targets selected from a Naïve Bayes (NB) classifier. The plurality of targets may comprise 2, 3, 4, 5, 6, 7, 8, 9, 10 or more targets selected from an NB classifier. The plurality of targets may comprise 12, 13, 14, 15, 17, 20, 22, 25 or more targets selected from an NB classifier. For example, a NB classifier of the present disclosure may comprise two or more targets selected from Table 5.

The one or more gene targets may comprise one or more targets selected from a recursive partitioning (Rpart) classifier. The plurality of targets may comprise 2, 3, 4, 5, 6, 7, 8, 9, 10 or more targets selected from an Rpart classifier. The plurality of targets may comprise 12, 13, 14, 15, 17, 20, 22, 25 or more targets selected from an Rpart classifier. For example, an Rpart classifier of the present disclosure may comprise two or more targets selected from Table 5.

The one or more gene targets may comprise one or more targets selected from a high dimensional discriminate analysis (HDDA) classifier. The plurality of targets may comprise two or more targets selected from a high dimensional discriminate analysis (HDDA) classifier. The plurality of targets may comprise three or more targets selected from a high dimensional discriminate analysis (HDDA) classifier. The plurality of targets may comprise 5, 6, 7, 8, 9, 10, 11, 12, 13, 14, 15, 17, 20, 22, 25 or more targets selected from a high dimensional discriminate analysis (HDDA) classifier. For example, an Rpart classifier of the present disclosure may comprise two or more targets selected from Table 5.

The present disclosure provides for a probe set for diagnosing, monitoring and/or predicting a status or outcome of breast cancer in a subject comprising a plurality of probes, wherein (i) the probes in the set are capable of detecting an expression level of at least one target; and (ii) the expression level determines the cancer status (e.g., risk of recurrence) of the subject with at least about 40% specificity.

The probe set may comprise one or more polynucleotide probes. Individual polynucleotide probes comprise a nucleotide sequence derived from the nucleotide sequence of the target sequences or complementary sequences thereof. The nucleotide sequence of the polynucleotide probe is designed such that it corresponds to, or is complementary to the target sequences. The polynucleotide probe can specifically hybridize under either stringent or lowered stringency hybridization conditions to a region of the target sequences, to the complement thereof, or to a nucleic acid sequence (such as a cDNA) derived therefrom.

The selection of the polynucleotide probe sequences and determination of their uniqueness may be carried out in silico using techniques known in the art, for example, based on a BLASTN search of the polynucleotide sequence in question against gene sequence databases, such as the Human Genome Sequence, UniGene, dbEST or the non-redundant database at NCBI. In one embodiment of the disclosure, the polynucleotide probe is complementary to a region of a target mRNA derived from a target sequence in the probe set. Computer programs can also be employed to select probe sequences that may not cross hybridize or may not hybridize non-specifically.

In some instances, microarray hybridization of RNA, extracted from breast cancer tissue samples and amplified, may yield a dataset that is then summarized and normalized by the fRMA technique. After removal (or filtration) of cross-hybridizing PSRs, and PSRs containing less than 4 probes, the remaining PSRs can be used in further analysis. Following fRMA and filtration, the data can be decomposed into its principal components and an analysis of variance model is used to determine the extent to which a batch effect remains present in the first 10 principal components.

These remaining PSRs can then be subjected to filtration by a T-test between CR (clinical recurrence) and non-CR samples. Using a p-value cut-off of 0.01, the remaining features (e.g., PSRs) can be further refined. Feature selection can be performed by regularized logistic regression using the elastic-net penalty. The regularized regression may be bootstrapped over 1000 times using all training data; with each iteration of bootstrapping, features that have non-zero co-efficient following 3-fold cross validation can be tabulated. In some instances, features that were selected in at least 25% of the total runs were used for model building.

The polynucleotide probes of the present disclosure may range in length from about 15 nucleotides to the full length of the coding target or non-coding target. In one embodiment of the disclosure, the polynucleotide probes are at least about 15 nucleotides in length. In another embodiment, the polynucleotide probes are at least about 20 nucleotides in length. In a further embodiment, the polynucleotide probes are at least about 25 nucleotides in length. In another embodiment, the polynucleotide probes are between about 15 nucleotides and about 500 nucleotides in length. In other embodiments, the polynucleotide probes are between about 15 nucleotides and about 450 nucleotides, about 15 nucleotides and about 400 nucleotides, about 15 nucleotides and about 350 nucleotides, about 15 nucleotides and about 300 nucleotides, about 15 nucleotides and about 250 nucleotides, about 15 nucleotides and about 200 nucleotides in length. In some embodiments, the probes are at least 15 nucleotides in length. In some embodiments, the probes are at least 15 nucleotides in length. In some embodiments, the probes are at least 20 nucleotides, at least 25 nucleotides, at least 50 nucleotides, at least 75 nucleotides, at least 100 nucleotides, at least 125 nucleotides, at least 150 nucleotides, at least 200 nucleotides, at least 225 nucleotides, at least 250 nucleotides, at least 275 nucleotides, at least 300 nucleotides, at least 325 nucleotides, at least 350 nucleotides, at least 375 nucleotides in length.

The polynucleotide probes of a probe set can comprise RNA, DNA, RNA or DNA mimetics, or combinations thereof, and can be single-stranded or double-stranded. Thus the polynucleotide probes can be composed of naturally-occurring nucleobases, sugars and covalent internucleoside (backbone) linkages as well as polynucleotide probes having non-naturally-occurring portions which function similarly. Such modified or substituted polynucleotide probes may provide desirable properties such as, for example, enhanced affinity for a target gene and increased stability. The probe set may comprise a coding target and/or a non-coding target.

In some embodiments, the probe set comprise a plurality of target sequences that hybridize to at least about 5 coding targets and/or non-coding targets. Alternatively, the probe set comprise a plurality of target sequences that hybridize to at least about 10 coding targets and/or non-coding targets. In some embodiments, the probe set comprise a plurality of target sequences that hybridize to at least about 15 coding targets and/or non-coding targets. In some embodiments, the probe set comprise a plurality of target sequences that hybridize to at least about 20 coding targets and/or non-coding targets. In some embodiments, the probe set comprise a plurality of target sequences that hybridize to at least about 30 coding targets and/or non-coding targets.

The system of the present disclosure further provides for primers and primer pairs capable of amplifying target sequences defined by the probe set, or fragments or subsequences or complements thereof. The nucleotide sequences of the probe set may be provided in computer-readable media for in silico applications and as a basis for the design of appropriate primers for amplification of one or more target sequences of the probe set.

Primers based on the nucleotide sequences of target sequences can be designed for use in amplification of the target sequences. For use in amplification reactions such as PCR, a pair of primers can be used. The exact composition of the primer sequences is not critical to the disclosure, but for most applications the primers may hybridize to specific sequences of the probe set under stringent conditions, particularly under conditions of high stringency, as known in the art. The pairs of primers are usually chosen so as to generate an amplification product of at least about 50 nucleotides, more usually at least about 100 nucleotides. Algorithms for the selection of primer sequences are generally known, and are available in commercial software packages. These primers may be used in standard quantitative or qualitative PCR-based assays to assess transcript expression levels of RNAs defined by the probe set. Alternatively, these primers may be used in combination with probes, such as molecular beacons in amplifications using real-time PCR.

In one embodiment, the primers or primer pairs, when used in an amplification reaction, specifically amplify at least a portion of a nucleic acid sequence of a target (or subgroups thereof as set forth herein), an RNA form thereof, or a complement to either thereof.

A label can optionally be attached to or incorporated into a probe or primer polynucleotide to allow detection and/or quantitation of a target polynucleotide representing the target sequence of interest. The target polynucleotide may be the expressed target sequence RNA itself, a cDNA copy thereof, or an amplification product derived therefrom, and may be the positive or negative strand, so long as it can be specifically detected in the assay being used. Similarly, an antibody may be labeled.

In certain multiplex formats, labels used for detecting different targets may be distinguishable. The label can be attached directly (e.g., via covalent linkage) or indirectly, e.g., via a bridging molecule or series of molecules (e.g., a molecule or complex that can bind to an assay component, or via members of a binding pair that can be incorporated into assay components, e.g. biotin-avidin or streptavidin). Many labels are commercially available in activated forms which can readily be used for such conjugation (for example through amine acylation), or labels may be attached through known or determinable conjugation schemes, many of which are known in the art.

Labels useful in the disclosure described herein include any substance which can be detected when bound to or incorporated into the biomolecule of interest. Any effective detection method can be used, including optical, spectroscopic, electrical, piezoelectrical, magnetic, Raman scattering, surface plasmon resonance, colorimetric, calorimetric, etc. A label is typically selected from a chromophore, a lumiphore, a fluorophore, one member of a quenching system, a chromogen, a hapten, an antigen, a magnetic particle, a material exhibiting nonlinear optics, a semiconductor nanocrystal, a metal nanoparticle, an enzyme, an antibody or binding portion or equivalent thereof, an aptamer, and one member of a binding pair, and combinations thereof. Quenching schemes may be used, wherein a quencher and a fluorophore as members of a quenching pair may be used on a probe, such that a change in optical parameters occurs upon binding to the target introduce or quench the signal from the fluorophore. One example of such a system is a molecular beacon. Suitable quencher/fluorophore systems are known in the art. The label may be bound through a variety of intermediate linkages. For example, a polynucleotide may comprise a biotin-binding species, and an optically detectable label may be conjugated to biotin and then bound to the labeled polynucleotide. Similarly, a polynucleotide sensor may comprise an immunological species such as an antibody or fragment, and a secondary antibody containing an optically detectable label may be added.

Chromophores useful in the methods described herein include any substance which can absorb energy and emit light. For multiplexed assays, a plurality of different signaling chromophores can be used with detectably different emission spectra. The chromophore can be a lumophore or a fluorophore. Typical fluorophores include fluorescent dyes, semiconductor nanocrystals, lanthanide chelates, polynucleotide-specific dyes and green fluorescent protein.

In some embodiments, polynucleotides of the disclosure comprise at least 20 consecutive bases of the nucleic acid sequence of a target or a complement thereto. The polynucleotides may comprise at least 21, 22, 23, 24, 25, 27, 30, 32, 35, 40, 45, 50, or more consecutive bases of the nucleic acids sequence of a target.

The polynucleotides may be provided in a variety of formats, including as solids, in solution, or in an array. The polynucleotides may optionally comprise one or more labels, which may be chemically and/or enzymatically incorporated into the polynucleotide.

In some embodiments, one or more polynucleotides provided herein can be provided on a substrate. The substrate can comprise a wide range of material, either biological, nonbiological, organic, inorganic, or a combination of any of these. For example, the substrate may be a polymerized Langmuir Blodgett film, functionalized glass, Si, Ge, GaAs, GaP, SiO₂, SiN₄, modified silicon, or any one of a wide variety of gels or polymers such as (poly)tetrafluoroethylene, (poly)vinylidenedifluoride, polystyrene, cross-linked polystyrene, polyacrylic, polylactic acid, polyglycolic acid, poly(lactide coglycolide), polyanhydrides, poly(methyl methacrylate), poly(ethylene-co-vinyl acetate), polysiloxanes, polymeric silica, latexes, dextran polymers, epoxies, polycarbonates, or combinations thereof. Conducting polymers and photoconductive materials can be used.

The substrate can take the form of an array, a photodiode, an optoelectronic sensor such as an optoelectronic semiconductor chip or optoelectronic thin-film semiconductor, or a biochip. The location(s) of probe(s) on the substrate can be addressable; this can be done in highly dense formats, and the location(s) can be microaddressable or nanoaddressable.

In certain embodiments, a sample (e.g., biological sample) containing breast cancer cells is collected from a subject in need of treatment for cancer to evaluate if the subject is at low risk or high risk of cancer recurrence based on an expression level or expression profile and likelihood of benefitting from adjuvant radiotherapy. Diagnostic samples for use with the systems and in the methods of the present disclosure comprise nucleic acids suitable for providing RNA expression information. In principle, the biological sample from which the expressed RNA is obtained and analyzed for target gene expression can be any material suspected of comprising cancerous breast tissue or cells. The diagnostic sample can be a biological sample used directly in a method of the disclosure. Alternatively, the diagnostic sample can be a sample prepared from a biological sample.

In one embodiment, the sample or portion of the sample comprising or suspected of comprising cancerous tissue or cells can be any source of biological material, including cells, tissue or fluid, including bodily fluids. Non-limiting examples of the source of the sample include an aspirate, a needle biopsy, a cytology pellet, a bulk tissue preparation or a section thereof obtained for example by surgery or autopsy, lymph fluid, blood, plasma, serum, tumors, and organs. In some embodiments, the sample is from a breast tumor biopsy.

The samples may be archival samples, having a known and documented medical outcome, or may be samples from current subjects whose ultimate medical outcome is not yet known.

In some embodiments, the sample may be dissected prior to molecular analysis. The sample may be prepared via macrodissection of a bulk tumor specimen or portion thereof, or may be treated via microdissection, for example via Laser Capture Microdissection (LCM).

The sample may initially be provided in a variety of states, as fresh tissue, fresh frozen tissue, fine needle aspirates, and may be fixed or unfixed. Frequently, medical laboratories routinely prepare medical samples in a fixed state, which facilitates tissue storage. A variety of fixatives can be used to fix tissue to stabilize the morphology of cells, and may be used alone or in combination with other agents. Exemplary fixatives include crosslinking agents, alcohols, acetone, Bouin's solution, Zenker solution, Hely solution, osmic acid solution and Camoy solution.

Crosslinking fixatives can comprise any agent suitable for forming two or more covalent bonds, for example an aldehyde. Sources of aldehydes typically used for fixation include formaldehyde, paraformaldehyde, glutaraldehyde or formalin. In some embodiments, the crosslinking agent comprises formaldehyde, which may be included in its native form or in the form of paraformaldehyde or formalin. One of skill in the art would appreciate that for samples in which crosslinking fixatives have been used special preparatory steps may be necessary including for example heating steps and proteinase-k digestion; see methods.

One or more alcohols may be used to fix tissue, alone or in combination with other fixatives. Exemplary alcohols used for fixation include methanol, ethanol and isopropanol.

Formalin fixation is frequently used in medical laboratories. Formalin comprises both an alcohol, typically methanol, and formaldehyde, both of which can act to fix a biological sample.

Whether fixed or unfixed, the biological sample may optionally be embedded in an embedding medium. Exemplary embedding media used in histology including paraffin, Tissue-Tek® V.I.P, Paramat, Paramat Extra, Paraplast, Paraplast X-tra, Paraplast Plus, Peel Away Paraffin Embedding Wax, Polyester Wax, Carbowax Polyethylene Glycol, Polyfin, Tissue Freezing Medium TFMFM, Cryo-Gef, and OCT Compound (Electron Microscopy Sciences, Hatfield, PA). Prior to molecular analysis, the embedding material may be removed via any suitable techniques, as known in the art. For example, where the sample is embedded in wax, the embedding material may be removed by extraction with organic solvent(s), for example xylenes. Kits are commercially available for removing embedding media from tissues. Samples or sections thereof may be subjected to further processing steps as needed, for example serial hydration or dehydration steps.

In some embodiments, the sample is a fixed, wax-embedded biological sample. Frequently, samples from medical laboratories are provided as fixed, wax-embedded samples, most commonly as formalin-fixed, paraffin embedded (FFPE) tissues.

Whatever the source of the biological sample, the target polynucleotide that is ultimately assayed can be prepared synthetically (in the case of control sequences), but typically is purified from the biological source and subjected to one or more preparative steps. The RNA may be purified to remove or diminish one or more undesired components from the biological sample or to concentrate it. Conversely, where the RNA is too concentrated for the particular assay, it may be diluted.

RNA can be extracted and purified from biological samples using any suitable technique. A number of techniques are known in the art, and several are commercially available (e.g., FormaPure nucleic acid extraction kit, Agencourt Biosciences, Beverly MA, High Pure FFPE RNA Micro Kit, Roche Applied Science, Indianapolis, IN). RNA can be extracted from frozen tissue sections using TRIzol (Invitrogen, Carlsbad, CA) and purified using RNeasy Protect kit (Qiagen, Valencia, CA). RNA can be further purified using DNAse I treatment (Ambion, Austin, TX) to eliminate any contaminating DNA. RNA concentrations can be made using a Nanodrop ND-1000 spectrophotometer (Nanodrop Technologies, Rockland, DE). RNA can be further purified to eliminate contaminants that interfere with cDNA synthesis by cold sodium acetate precipitation. RNA integrity can be evaluated by running electropherograms, and RNA integrity number (RIN, a correlative measure that indicates intactness of mRNA) can be determined using the RNA 6000 PicoAssay for the Bioanalyzer 2100 (Agilent Technologies, Santa Clara, CA).

Kits for performing the desired method(s) are also provided, and comprise a container or housing for holding the components of the kit, one or more vessels containing one or more nucleic acid(s), and optionally one or more vessels containing one or more reagents. The reagents include those described in the composition of matter section above, and those reagents useful for performing the methods described, including amplification reagents, and may include one or more probes, primers or primer pairs, enzymes (including polymerases and ligases), intercalating dyes, labeled probes, and labels that can be incorporated into amplification products.

In some embodiments, the kit comprises primers or primer pairs specific for those subsets and combinations of target sequences described herein. The primers or pairs of primers are suitable for selectively amplifying the target sequences. The kit may comprise at least two, three, four or five primers or pairs of primers suitable for selectively amplifying one or more targets. The kit may comprise at least 5, 10, 15, 20, 30, 40, 50, 60, 70, 80, 90, 100, 110, or more primers or pairs of primers suitable for selectively amplifying one or more targets.

In some embodiments, the primers or primer pairs of the kit, when used in an amplification reaction, specifically amplify a non-coding target, coding target, exonic, or non-exonic target described herein, a nucleic acid sequence corresponding to a target selected from Table 5, an RNA form thereof, or a complement to either thereof. The kit may include a plurality of such primers or primer pairs which can specifically amplify a corresponding plurality of different amplify a non-coding target, coding target, exonic, or non-exonic transcript described herein, a nucleic acid sequence corresponding to a target selected from Table 5, RNA forms thereof, or complements thereto. At least two, three, four or five primers or pairs of primers suitable for selectively amplifying the one or more targets can be provided in kit form. In some embodiments, the kit comprises from five to fifty primers or pairs of primers suitable for amplifying the one or more targets.

The reagents may independently be in liquid or solid form. The reagents may be provided in mixtures. Control samples and/or nucleic acids may optionally be provided in the kit. Control samples may include tissue and/or nucleic acids obtained from or representative of tumor samples from subjects showing no evidence of disease, as well as tissue and/or nucleic acids obtained from or representative of tumor samples from subjects that develop systemic cancer.

The nucleic acids may be provided in an array format, and thus an array or microarray may be included in the kit. The kit optionally may be certified by a government agency for use in prognosing the disease outcome of cancer subjects and/or for designating a treatment modality.

Instructions for using the kit to perform one or more methods of the disclosure can be provided with the container, and can be provided in any fixed medium. The instructions may be located inside or outside the container or housing, and/or may be printed on the interior or exterior of any surface thereof. A kit may be in multiplex form for concurrently detecting and/or quantitating one or more different target polynucleotides representing the expressed target genes.

Following sample collection and nucleic acid extraction, the nucleic acid portion of the sample comprising RNA that is or can be used to prepare the target polynucleotide(s) of interest can be subjected to one or more preparative reactions. These preparative reactions can include in vitro transcription (IVT), labeling, fragmentation, amplification and other reactions. mRNA can first be treated with reverse transcriptase and a primer to create cDNA prior to detection, quantitation and/or amplification; this can be done in vitro with purified mRNA or in situ, e.g., in cells or tissues affixed to a slide.

By “amplification” is meant any process of producing at least one copy of a nucleic acid, such as an expressed RNA, and in many cases produces multiple copies. An amplification product can be RNA or DNA, and may include a complementary strand to the expressed target sequence. DNA amplification products can be produced initially through reverse translation and then optionally from further amplification reactions. The amplification product may include all or a portion of a target sequence, and may optionally be labeled. A variety of amplification methods are suitable for use, including polymerase-based methods and ligation-based methods. Exemplary amplification techniques include the polymerase chain reaction method (PCR), the lipase chain reaction (LCR), ribozyme-based methods, self-sustained sequence replication (3SR), nucleic acid sequence-based amplification (NASBA), the use of Q Beta replicase, reverse transcription, nick translation, and the like.

Asymmetric amplification reactions may be used to preferentially amplify one strand representing the target sequence that is used for detection as the target polynucleotide. In some cases, the presence and/or amount of the amplification product itself may be used to determine the expression level of a given target sequence. In other instances, the amplification product may be used to hybridize to an array or other substrate comprising sensor polynucleotides which are used to detect and/or quantitate target sequence expression.

The first cycle of amplification in polymerase-based methods typically forms a primer extension product complementary to the template strand. If the template is single-stranded RNA, a polymerase with reverse transcriptase activity is used in the first amplification to reverse transcribe the RNA to DNA, and additional amplification cycles can be performed to copy the primer extension products. The primers for a PCR must, of course, be designed to hybridize to regions in their corresponding template that can produce an amplifiable segment; thus, each primer must hybridize so that its 3′ nucleotide is paired to a nucleotide in its complementary template strand that is located 3′ from the 3′ nucleotide of the primer used to replicate that complementary template strand in the PCR.

The target polynucleotide can be amplified by contacting one or more strands of the target polynucleotide with a primer and a polymerase having suitable activity to extend the primer and copy the target polynucleotide to produce a full-length complementary polynucleotide or a smaller portion thereof. Any enzyme having a polymerase activity that can copy the target polynucleotide can be used, including DNA polymerases, RNA polymerases, reverse transcriptases, enzymes having more than one type of polymerase or enzyme activity. The enzyme can be thermolabile or thermostable. Mixtures of enzymes can also be used. Exemplary enzymes include: DNA polymerases such as DNA Polymerase I (“Pol I”), the Klenow fragment of Pol I, T4, T7, Sequenase® T7, Sequenase® Version 2.0 T7, Tub, Taq, Tth, Pfic, Pfu, Tsp, Tfl, Tli and Pyrococcus sp GB-D DNA polymerases; RNA polymerases such as E. coil, SP6, T3 and T7 RNA polymerases; and reverse transcriptases such as AMV, M-MuLV, MMLV, RNAse H MMLV (SuperScript®), SuperScript® II, ThermoScript®, HIV-1, and RAV2 reverse transcriptases. All of these enzymes are commercially available. Exemplary polymerases with multiple specificities include RAV2 and Tli (exo-) polymerases. Exemplary thermostable polymerases include Tub, Taq, Tth, Pfic, Pfu, Tsp, Tfl, Tli and Pyrococcus sp. GB-D DNA polymerases.

Suitable reaction conditions are chosen to permit amplification of the target polynucleotide, including pH, buffer, ionic strength, presence and concentration of one or more salts, presence and concentration of reactants and cofactors such as nucleotides and magnesium and/or other metal ions (e.g., manganese), optional cosolvents, temperature, thermal cycling profile for amplification schemes comprising a polymerase chain reaction, and may depend in part on the polymerase being used as well as the nature of the sample. Cosolvents include formamide (typically at from about 2 to about 10%), glycerol (typically at from about 5 to about 10%), and DMSO (typically at from about 0.9 to about 10%). Techniques may be used in the amplification scheme in order to minimize the production of false positives or artifacts produced during amplification. These include “touchdown” PCR, hot-start techniques, use of nested primers, or designing PCR primers so that they form stem-loop structures in the event of primer-dimer formation and thus are not amplified. Techniques to accelerate PCR can be used, for example centrifugal PCR, which allows for greater convection within the sample, and comprising infrared heating steps for rapid heating and cooling of the sample. One or more cycles of amplification can be performed. An excess of one primer can be used to produce an excess of one primer extension product during PCR. In some embodiments, the primer extension product produced in excess is the amplification product to be detected. A plurality of different primers may be used to amplify different target polynucleotides or different regions of a particular target polynucleotide within the sample.

An amplification reaction can be performed under conditions which allow an optionally labeled sensor polynucleotide to hybridize to the amplification product during at least part of an amplification cycle. When the assay is performed in this manner, real-time detection of this hybridization event can take place by monitoring for light emission or fluorescence during amplification, as known in the art.

Where the amplification product is to be used for hybridization to an array or microarray, a number of suitable commercially available amplification products are available. These include amplification kits available from NuGEN, Inc. (San Carlos, CA), including the WT-Ovation™ System, WT-Ovation™ System v2, WT-Ovation™ Pico System, WT-Ovation™ FFPE Exon Module, WT-Ovation™ FFPE Exon Module RiboAmp and RiboAmp^{Plus} RNA Amplification Kits (MDS Analytical Technologies (formerly Arcturus) (Mountain View, CA), Genisphere, Inc. (Hatfield, PA), including the RampUp Plus and SenseAmp RNA Amplification kits, alone or in combination. Amplified nucleic acids may be subjected to one or more purification reactions after amplification and labeling, for example using magnetic beads (e.g., RNAClean magnetic beads, Agencourt Biosciences).

Multiple RNA biomarkers can be analyzed using real-time quantitative multiplex RT-PCR platforms and other multiplexing technologies such as GenomeLab GeXP Genetic Analysis System (Beckman Coulter, Foster City, CA), SmartCycler® 9600 or GeneXpert® Systems (Cepheid, Sunnyvale, CA), ABI 7900 HT Fast Real Time PCR system (Applied Biosystems, Foster City, CA), LightCycler® 480 System (Roche Molecular Systems, Pleasanton, CA), xMAP 100 System (Luminex, Austin, TX) Solexa Genome Analysis System (Illumina, Hayward, CA), OpenArray Real Time qPCR (BioTrove, Woburn, MA) and BeadXpress System (Illumina, Hayward, CA).

Any method of detecting and/or quantitating the expression of the encoded target genes can in principle be used in the disclosure. The expressed target genes can be directly detected and/or quantitated, or may be copied and/or amplified to allow detection of amplified copies of the expressed target genes.

Methods for detecting and/or quantifying a target gene can include Northern blotting, sequencing, array or microarray hybridization, by enzymatic cleavage of specific structures (e.g., a Clariom S assay, ThermoFisher Scientific, an Invader® assay, Third Wave Technologies, e.g. as described in U.S. Pat. Nos. 5,846,717, 6,090,543; 6,001,567; 5,985,557; and 5,994,069) and amplification methods, e.g. RT-PCR, including in a TaqMan® assay (PE Biosystems, Foster City, Calif., e.g. as described in U.S. Pat. Nos. 5,962,233 and 5,538,848), and may be quantitative or semi-quantitative, and may vary depending on the origin, amount and condition of the available biological sample. Combinations of these methods may also be used. For example, nucleic acids may be amplified, labeled and subjected to microarray analysis. Methods for detecting and/or quantifying a target gene can include gene-level expression analysis of annotated genes using microarray hybridization (e.g., GeneChip Human Exon 1.0 ST assay or Clariom S assay, ThermoFisher Scientific).

In some instances, target genes may be detected by sequencing. Sequencing methods may comprise whole genome sequencing or exome sequencing. Sequencing methods such as Maxim-Gilbert, chain-termination, or high-throughput systems may also be used. Additional, suitable sequencing techniques include classic dideoxy sequencing reactions (Sanger method) using labeled terminators or primers and gel separation in slab or capillary, sequencing by synthesis using reversibly terminated labeled nucleotides, pyrosequencing, 454 sequencing, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, and SOLiD sequencing.

Additional methods for detecting and/or quantifying a target gene include single-molecule sequencing (e.g., Helicos, PacBio), sequencing by synthesis (e.g., Illumina, Ion Torrent), sequencing by ligation (e.g., ABI SOLID), sequencing by hybridization (e.g., Complete Genomics), in situ hybridization, bead-array technologies (e.g., Luminex xMAP, Illumina BeadChips), branched DNA technology (e.g., Panomics, Genisphere). Sequencing methods may use fluorescent (e.g., Illumina) or electronic (e.g., Ion Torrent, Oxford Nanopore) methods of detecting nucleotides.

Reverse transcription can be performed by any method known in the art. For example, reverse transcription may be performed using the Omniscript kit (Qiagen, Valencia, CA), Superscript III kit (Invitrogen, Carlsbad, CA), for RT-PCR. Target-specific priming can be performed in order to increase the sensitivity of detection of target genes and generate target-specific cDNA.

TaqMan® RT-PCR can be performed using Applied Biosystems Prism (ABI) 7900 HT instruments in a 5 1.11 volume with target gene-specific cDNA equivalent to 1 ng total RNA.

Primers and probes concentrations for TaqMan analysis are added to amplify fluorescent amplicons using PCR cycling conditions such as 95° C. for 10 minutes for one cycle, 95° C. for 20 seconds, and 60° C. for 45 seconds for 40 cycles. A reference sample can be assayed to ensure reagent and process stability. Negative controls (e.g., no template) should be assayed to monitor any exogenous nucleic acid contamination.

The present disclosure contemplates that a probe set or probes derived therefrom may be provided in an array format. In the context of the present disclosure, an “array” is a spatially or logically organized collection of polynucleotide probes. An array comprising probes specific for a coding target, non-coding target, or a combination thereof may be used. Alternatively, an array comprising probes specific for two or more of transcripts of a target, or a product derived thereof, can be used. Desirably, an array may be specific for 5, 10, 15, 20, 25, 30 or more of transcripts of a target gene. Expression of these genes may be detected alone or in combination with other transcripts. In some embodiments, an array is used which comprises a wide range of sensor probes for breast-specific expression products, along with appropriate control sequences. In some instances, the array may comprise the Human Exon 1.0 ST Array (HuEx 1.0 ST, Thermo Fisher Scientific, Santa Clara, CA.).

Typically the polynucleotide probes are attached to a solid substrate and are ordered so that the location (on the substrate) and the identity of each are known. The polynucleotide probes can be attached to one of a variety of solid substrates capable of withstanding the reagents and conditions necessary for use of the array. Examples include, but are not limited to, polymers, such as (poly)tetrafluoroethylene, (poly)vinylidenedifluoride, polystyrene, polycarbonate, polypropylene and polystyrene; ceramic; silicon; silicon dioxide; modified silicon; (fused) silica, quartz or glass; functionalized glass; paper, such as filter paper; diazotized cellulose; nitrocellulose filter; nylon membrane; and polyacrylamide gel pad. Substrates that are transparent to light are useful for arrays that may be used in an assay that involves optical detection.

Examples of array formats include membrane or filter arrays (for example, nitrocellulose, nylon arrays), plate arrays (for example, multiwell, such as a 24-, 96-, 256-, 384-, 864- or 1536-well, microtitre plate arrays), pin arrays, and bead arrays (for example, in a liquid “slurry”). Arrays on substrates such as glass or ceramic slides are often referred to as chip arrays or “chips.” Such arrays are well known in the art. In one embodiment of the present disclosure, the Cancer Prognosticarray is a chip.

In some embodiments, one or more pattern recognition methods can be used in analyzing the expression level of target genes. The pattern recognition method can comprise a linear combination of expression levels, or a nonlinear combination of expression levels. In some embodiments, expression measurements for RNA transcripts or combinations of RNA transcript levels are formulated into linear or non-linear models or algorithms (e.g., an ‘expression signature’) and converted into a likelihood score. This likelihood score indicates the probability that a biological sample is from a subject who may exhibit no evidence of disease, who may exhibit systemic cancer, or who may exhibit biochemical recurrence or locoregional recurrence. The likelihood score can be used to distinguish these disease states. The models and/or algorithms can be provided in machine readable format, and may be used to correlate expression levels or an expression profile with a disease state, and/or to designate a treatment modality for a subject or class of subjects.

Assaying the expression level for a plurality of target genes may comprise the use of an algorithm or classifier. Array data can be managed, classified, and analyzed using techniques known in the art. Assaying the expression level for a plurality of gene targets may comprise probe set modeling and data pre-processing. Probe set modeling and data pre-processing can be derived using the Robust Multi-Array (RMA) algorithm or variants GC-RMA, fRMA, Probe Logarithmic Intensity Error (PLIER) algorithm or variant iterPLIER. Variance or intensity filters can be applied to pre-process data using the RMA algorithm, for example by removing target genes with a standard deviation of <10 or a mean intensity of <100 intensity units of a normalized data range, respectively.

Alternatively, assaying the expression level for a plurality of gene targets may comprise the use of a machine learning algorithm. The machine learning algorithm may comprise a supervised learning algorithm. Examples of supervised learning algorithms may include Average One-Dependence Estimators (AODE), Artificial neural network (e.g., Backpropagation), Bayesian statistics (e.g., Naive Bayes classifier, Bayesian network, Bayesian knowledge base), Case-based reasoning, Decision trees, Inductive logic programming, Gaussian process regression, Group method of data handling (GMDH), Learning Automata, Learning Vector Quantization, Minimum message length (decision trees, decision graphs, etc.), Lazy learning, Instance-based learning Nearest Neighbor Algorithm, Analogical modeling, Probably approximately correct learning (PAC) learning, Ripple down rules, a knowledge acquisition methodology, Symbolic machine learning algorithms, Subsymbolic machine learning algorithms, Support vector machines, Random Forests, Ensembles of classifiers, Bootstrap aggregating (bagging), and Boosting. Supervised learning may comprise ordinal classification such as regression analysis and Information fuzzy networks (IFN). Alternatively, supervised learning methods may comprise statistical classification, such as AODE, Linear classifiers (e.g., Fishers linear discriminant, Logistic regression, Naive Bayes classifier, Perceptron, and Support vector machine, Lasso and Elastic-Net Regularized General Linear models), quadratic classifiers, k-nearest neighbor, Boosting, Decision trees (e.g., C4.5, Random forests), Bayesian networks, and Hidden Markov models.

The machine learning algorithms may also comprise an unsupervised learning algorithm. Examples of unsupervised learning algorithms may include artificial neural network, Data clustering, Expectation-maximization algorithm, Self-organizing map, Radial basis function network, Vector Quantization, Generative topographic map, Information bottleneck method, and IBSEAD. Unsupervised learning may also comprise association rule learning algorithms such as Apriori algorithm, Eclat algorithm and FP-growth algorithm. Hierarchical clustering, such as Single-linkage clustering and Conceptual clustering, may also be used. Alternatively, unsupervised learning may comprise partitional clustering such as K-means algorithm and Fuzzy clustering.

In some instances, the machine learning algorithms comprise a reinforcement learning algorithm. Examples of reinforcement learning algorithms include, but are not limited to, temporal difference learning, Q-learning and Learning Automata. Alternatively, the machine learning algorithm may comprise Data Pre-processing.

In some embodiments, the machine learning algorithms may include, but are not limited to, Average One-Dependence Estimators (AODE), Fisher's linear discriminant, Logistic regression, Perceptron, Multilayer Perceptron, Artificial Neural Networks, Support vector machines, Quadratic classifiers, Boosting, Decision trees, C4.5, Bayesian networks, Hidden Markov models, High-Dimensional Discriminant Analysis, and Gaussian Mixture Models. The machine learning algorithm may comprise support vector machines, Naïve Bayes classifier, k-nearest neighbor, high-dimensional discriminant analysis, or Gaussian mixture models. In some instances, the machine learning algorithm comprises Random Forests.

Molecular subtyping is a method of classifying breast cancer into one of multiple genetically-distinct categories, or subtypes. Each subtype responds differently to different kinds of treatments, and the presence of a particular subtype is predictive of, for example, radioresistance or chemoresistance, higher or lower risk of recurrence, or good or poor prognosis for an individual. Differential expression analysis of a plurality of the gene targets listed in Table 5 allows for the identification of subjects at low risk of cancer recurrence who, for example, may benefit least from adjuvant radiotherapy. In some instances, the molecular subtyping methods of the present disclosure are used in combination with other biomarkers, like tumor grade and hormone levels, for analyzing the breast cancer. For example, a subject with estrogen receptor positive (ER+), human epidermal growth factor receptor 2 negative (HER2−) breast cancer, node-negative breast cancer, who is post-menopausal, may be more likely to have a lower risk of recurrence (e.g., locoregional recurrence).

Cancer recurrence is the return of cancer after a period when no cancer cells are detected in the body. Following surgery for operable breast cancer, disease can recur locally, regionally, and/or at distant metastatic sites. A local recurrence is the reappearance of cancer on the ipsilateral chest wall. In contrast, a regional recurrence denotes tumor involving the regional lymph nodes, usually ipsilateral axillary or supraclavicular, and less commonly infraclavicular and/or internal mammary nodes. Some breast cancer patients will have local or locoregional recurrence after breast-conserving surgery and radiotherapy within ten years of first being diagnosed with breast cancer. If the breast was removed in the course of initial treatment, these women will have a local recurrence in the armpit or the chest wall within ten years. In some embodiments, the subject treated in the methods of the present disclosure has a node-negative breast cancer.

Diagnosing, predicting, or monitoring a status or outcome of a cancer may comprise treating a cancer or preventing a cancer progression. In addition, diagnosing, predicting, or monitoring a status or outcome of a cancer may comprise identifying or predicting that a subject is at low or high risk of a recurrence (e.g., locoregional recurrence). In some instances, diagnosing, predicting, or monitoring may comprise determining a therapeutic regimen. Determining a therapeutic regimen may comprise administering an anti-cancer therapy. Alternatively, determining a therapeutic regimen may comprise modifying, recommending, continuing or discontinuing an anti-cancer regimen. For example, a subject determined to be at low risk of recurrence of breast cancer based on expression profiling, as described herein, may be spared adjuvant radiotherapy. In some instances, a subject determined to be at high risk of recurrence of breast cancer based on expression profiling, as described herein, may be treated with mastectomy, radiation boost, or adjuvant systemic therapy. In some instances, if the sample expression patterns are consistent with the expression pattern for a known disease or disease outcome, the expression patterns can be used to designate one or more treatment modalities (e.g., therapeutic regimens, anti-cancer regimen). An anti-cancer regimen may comprise one or more anti-cancer therapies. Examples of anti-cancer therapies include hormonal/endocrine therapy, surgery, chemotherapy, radiation therapy, immunotherapy/biological therapy, and photodynamic therapy.

Hormonal therapy or endocrine therapy may involve administration of hormones, such as steroid hormones or hormone antagonists to modulate the levels of certain hormones in order to arrest growth or induce apoptosis of hormone-responsive cancer cells. For example, breast cancer may be treated with a selective estrogen receptor modulator (SERM) such as, but not limited to, tamoxifen, raloxifene, and toremifene. Alternately or additionally, inhibitors of hormone synthesis such as aromatase inhibitors, including, but not limited to, letrozole, anastrozole, exemestane, and aminoglutethimide may be used to treat breast cancer. In some cases, hormone supplementation with progestins such as, but not limited to, megestrol acetate and medroxyprogesterone acetate may be used for the treatment of hormone-responsive, advanced breast cancer. In particular, ER+breast cancer can be treated with either an estrogen receptor antagonist (e.g. tamoxifen) or a drug that blocks the production of estrogen such as an aromatase inhibitor (e.g. anastrozole or letrozole). Hormonal therapy may also include surgical removal of endocrine organs (e.g., orchiectomy or oophorectomy).

Surgical oncology uses surgical methods to diagnose, stage, and treat cancer, and to relieve certain cancer-related symptoms. Surgery may be used to remove the tumor (e.g., excisions, resections, debulking surgery), reconstruct a part of the body (e.g., restorative surgery), and/or to relieve symptoms such as pain (e.g., palliative surgery). Surgery may also include cryosurgery. Cryosurgery (also called cryotherapy) may use extreme cold produced by liquid nitrogen (or argon gas) to destroy abnormal tissue. Cryosurgery can be used to treat external tumors, such as those on the skin. For external tumors, liquid nitrogen can be applied directly to the cancer cells with a cotton swab or spraying device. Cryosurgery may also be used to treat tumors inside the body (internal tumors and tumors in the bone). For internal tumors, liquid nitrogen or argon gas may be circulated through a hollow instrument called a cryoprobe, which is placed in contact with the tumor. An ultrasound or MRI may be used to guide the cryoprobe and monitor the freezing of the cells, thus limiting damage to nearby healthy tissue. A ball of ice crystals may form around the probe, freezing nearby cells. Sometimes more than one probe is used to deliver the liquid nitrogen to various parts of the tumor. The probes may be put into the tumor during surgery or through the skin (percutaneously). After cryosurgery, the frozen tissue thaws and may be naturally absorbed by the body (for internal tumors), or may dissolve and form a scab (for external tumors).

Chemotherapeutic agents may also be used for the treatment of cancer. Examples of chemotherapeutic agents include alkylating agents, anti-metabolites, plant alkaloids and terpenoids, vinca alkaloids, podophyllotoxin, taxanes, topoisomerase inhibitors, and cytotoxic antibiotics. Cisplatin, carboplatin, and oxaliplatin are examples of alkylating agents. Other alkylating agents include mechlorethamine, cyclophosphamide, chlorambucil, ifosfamide. Alkylating agents may impair cell function by forming covalent bonds with the amino, carboxyl, sulfhydryl, and phosphate groups in biologically important molecules. Alternatively, alkylating agents may chemically modify a cell's DNA.

Anti-metabolites are another example of chemotherapeutic agents. Anti-metabolites may masquerade as purines or pyrimidines and may prevent purines and pyrimidines from becoming incorporated in to DNA during the “S” phase (of the cell cycle), thereby stopping normal development and division. Antimetabolites may also affect RNA synthesis. Examples of metabolites include azathioprine and mercaptopurine.

Alkaloids may be derived from plants and block cell division may also be used for the treatment of cancer. Alkyloids may prevent microtubule function. Examples of alkaloids are vinca alkaloids and taxanes. Vinca alkaloids may bind to specific sites on tubulin and inhibit the assembly of tubulin into microtubules (M phase of the cell cycle). The vinca alkaloids may be derived from the Madagascar periwinkle, Catharanthus roseus (formerly known as Vinca rosea). Examples of vinca alkaloids include, but are not limited to, vincristine, vinblastine, vinorelbine, or vindesine. Taxanes are diterpenes produced by the plants of the genus Taxus (yews). Taxanes may be derived from natural sources or synthesized artificially. Taxanes include paclitaxel (Taxol) and docetaxel (Taxotere). Taxanes may disrupt microtubule function. Microtubules are essential to cell division, and taxanes may stabilize GDP-bound tubulin in the microtubule, thereby inhibiting the process of cell division. Thus, in essence, taxanes may be mitotic inhibitors. Taxanes may also be radiosensitizing and often contain numerous chiral centers.

Alternative chemotherapeutic agents include podophyllotoxin. Podophyllotoxin is a plant-derived compound that may help with digestion and may be used to produce cytostatic drugs such as etoposide and teniposide. They may prevent the cell from entering the G1 phase (the start of DNA replication) and the replication of DNA (the S phase).

Topoisomerases are essential enzymes that maintain the topology of DNA. Inhibition of type I or type II topoisomerases may interfere with both transcription and replication of DNA by upsetting proper DNA supercoiling. Some chemotherapeutic agents may inhibit topoisomerases. For example, some type I topoisomerase inhibitors include camptothecins: irinotecan and topotecan. Examples of type II inhibitors include amsacrine, etoposide, etoposide phosphate, and teniposide. Kinase inhibitors may be used to treat breast cancer.

Another example of chemotherapeutic agents is cytotoxic antibiotics. Cytotoxic antibiotics are a group of antibiotics that are used for the treatment of cancer because they may interfere with DNA replication and/or protein synthesis. Cytotoxic antiobiotics include, but are not limited to, actinomycin, anthracyclines, doxorubicin, daunorubicin, valrubicin, idarubicin, epirubicin, bleomycin, plicamycin, and mitomycin.

In some instances, the anti-cancer treatment may comprise radiation therapy. Radiation can come from a machine outside the body (external-beam radiation therapy) or from radioactive material placed in the body near cancer cells (internal radiation therapy, more commonly called brachytherapy). Systemic radiation therapy uses a radioactive substance, given by mouth or into a vein that travels in the blood to tissues throughout the body.

External-beam radiation therapy may be delivered in the form of photon beams (either x-rays or gamma rays). A photon is the basic unit of light and other forms of electromagnetic radiation. An example of external-beam radiation therapy is called 3-dimensional conformal radiation therapy (3D-CRT). 3D-CRT may use computer software and advanced treatment machines to deliver radiation to very precisely shaped target areas. Many other methods of external-beam radiation therapy are currently being tested and used in cancer treatment. These methods include, but are not limited to, intensity-modulated radiation therapy (IMRT), image-guided radiation therapy (IGRT), Stereotactic radiosurgery (SRS), Stereotactic body radiation therapy (SBRT), and proton therapy.

Intensity-modulated radiation therapy (IMRT) is an example of external-beam radiation and may use hundreds of tiny radiation beam-shaping devices, called collimators, to deliver a single dose of radiation. The collimators can be stationary or can move during treatment, allowing the intensity of the radiation beams to change during treatment sessions. This kind of dose modulation allows different areas of a tumor or nearby tissues to receive different doses of radiation. IMRT is planned in reverse (called inverse treatment planning). In inverse treatment planning, the radiation doses to different areas of the tumor and surrounding tissue are planned in advance, and then a high-powered computer program calculates the required number of beams and angles of the radiation treatment. In contrast, during traditional (forward) treatment planning, the number and angles of the radiation beams are chosen in advance and computers calculate how much dose may be delivered from each of the planned beams. The goal of IMRT is to increase the radiation dose to the areas that need it and reduce radiation exposure to specific sensitive areas of surrounding normal tissue.

Another example of external-beam radiation is image-guided radiation therapy (IGRT). In IGRT, repeated imaging scans (CT, MRI, or PET) may be performed during treatment. These imaging scans may be processed by computers to identify changes in a tumor's size and location due to treatment and to allow the position of the subject or the planned radiation dose to be adjusted during treatment as needed. Repeated imaging can increase the accuracy of radiation treatment and may allow reductions in the planned volume of tissue to be treated, thereby decreasing the total radiation dose to normal tissue.

Tomotherapy is a type of image-guided IMRT. A tomotherapy machine is a hybrid between a CT imaging scanner and an external-beam radiation therapy machine. The part of the tomotherapy machine that delivers radiation for both imaging and treatment can rotate completely around the subject in the same manner as a normal CT scanner. Tomotherapy machines can capture CT images of the subject's tumor immediately before treatment sessions, to allow for very precise tumor targeting and sparing of normal tissue.

Stereotactic radiosurgery (SRS) can deliver one or more high doses of radiation to a small tumor. SRS uses extremely accurate image-guided tumor targeting and subject positioning. Therefore, a high dose of radiation can be given without excess damage to normal tissue. SRS can be used to treat small tumors with well-defined edges. It is most commonly used in the treatment of brain or spinal tumors and brain metastases from other cancer types. For the treatment of some brain metastases, subjects may receive radiation therapy to the entire brain (called whole-brain radiation therapy) in addition to SRS. SRS requires the use of a head frame or other device to immobilize the subject during treatment to ensure that the high dose of radiation is delivered accurately.

Stereotactic body radiation therapy (SBRT) delivers radiation therapy in fewer sessions, using smaller radiation fields and higher doses than 3D-CRT in most cases. SBRT may treat tumors that lie outside the brain and spinal cord. Because these tumors are more likely to move with the normal motion of the body, and therefore cannot be targeted as accurately as tumors within the brain or spine, SBRT is usually given in more than one dose. SBRT can be used to treat small, isolated tumors, including cancers in the lung and liver. SBRT systems may be known by their brand names, such as the CyberKnife®.

In proton therapy, external-beam radiation therapy may be delivered by proton. Protons are a type of charged particle. Proton beams differ from photon beams mainly in the way they deposit energy in living tissue. Whereas photons deposit energy in small packets all along their path through tissue, protons deposit much of their energy at the end of their path (called the Bragg peak) and deposit less energy along the way. Use of protons may reduce the exposure of normal tissue to radiation, possibly allowing the delivery of higher doses of radiation to a tumor.

Other charged particle beams such as electron beams may be used to irradiate superficial tumors, such as skin cancer or tumors near the surface of the body, but they cannot travel very far through tissue.

Internal radiation therapy (brachytherapy) is radiation delivered from radiation sources (radioactive materials) placed inside or on the body. Several brachytherapy techniques are used in cancer treatment. Interstitial brachytherapy may use a radiation source placed within tumor tissue, such as within a breast tumor. Intracavitary brachytherapy may use a source placed within a surgical cavity or a body cavity, such as the chest cavity, near a tumor. Episcleral brachytherapy, which may be used to treat melanoma inside the eye, may use a source that is attached to the eye. In brachytherapy, radioactive isotopes can be sealed in tiny pellets or “seeds.” These seeds may be placed in subjects using delivery devices, such as needles, catheters, or some other type of carrier. As the isotopes decay naturally, they give off radiation that may damage nearby cancer cells. Brachytherapy may be able to deliver higher doses of radiation to some cancers than external-beam radiation therapy while causing less damage to normal tissue.

Brachytherapy can be given as a low-dose-rate or a high-dose-rate treatment. In low-dose-rate treatment, cancer cells receive continuous low-dose radiation from the source over a period of several days. In high-dose-rate treatment, a robotic machine attached to delivery tubes placed inside the body may guide one or more radioactive sources into or near a tumor, and then removes the sources at the end of each treatment session. High-dose-rate treatment can be given in one or more treatment sessions. An example of a high-dose-rate treatment is the MammoSite® system. Bracytherapy may be used to treat subjects with breast cancer who have undergone breast-conserving surgery.

The placement of brachytherapy sources can be temporary or permanent. For permanent brachytherapy, the sources may be surgically sealed within the body and left there, even after all of the radiation has been given off. In some instances, the remaining material (in which the radioactive isotopes were sealed) does not cause any discomfort or harm to the subject. Permanent brachytherapy is a type of low-dose-rate brachytherapy. For temporary brachytherapy, tubes (catheters) or other carriers are used to deliver the radiation sources, and both the carriers and the radiation sources are removed after treatment. Temporary brachytherapy can be either low-dose-rate or high-dose-rate treatment. Brachytherapy may be used alone or in addition to external-beam radiation therapy to provide a “boost” of radiation to a tumor while sparing surrounding normal tissue.

In systemic radiation therapy, a subject may swallow or receive an injection of a radioactive substance, such as radioactive iodine or a radioactive substance bound to a monoclonal antibody. Radioactive iodine (131I) is a type of systemic radiation therapy commonly used to help treat cancer, such as thyroid cancer. Thyroid cells naturally take up radioactive iodine. For systemic radiation therapy for some other types of cancer, a monoclonal antibody may help target the radioactive substance to the right place. The antibody joined to the radioactive substance travels through the blood, locating and killing tumor cells. For example, the drug ibritumomab tiuxetan (Zevalin®) may be used for the treatment of certain types of B-cell non-Hodgkin lymphoma (NHL). The antibody part of this drug recognizes and binds to a protein found on the surface of B lymphocytes. The combination drug regimen of tositumomab and iodine I 131 tositumomab (Bexxar®) may be used for the treatment of certain types of cancer, such as NHL. In this regimen, nonradioactive tositumomab antibodies may be given to subjects first, followed by treatment with tositumomab antibodies that have 131I attached. Tositumomab may recognize and bind to the same protein on B lymphocytes as ibritumomab. The nonradioactive form of the antibody may help protect normal B lymphocytes from being damaged by radiation from 131I.

Some systemic radiation therapy drugs relieve pain from cancer that has spread to the bone (bone metastases). This is a type of palliative radiation therapy. The radioactive drugs samarium-153-lexidronam (Quadramet®) and strontium-89 chloride (Metastron®) are examples of radiopharmaceuticals may be used to treat pain from bone metastases.

Biological therapy (sometimes called immunotherapy, biotherapy, biologic therapy, or biological response modifier (BRM) therapy) uses the body's immune system, either directly or indirectly, to fight cancer or to lessen the side effects that may be caused by some cancer treatments. Biological therapies include interferons, interleukins, colony-stimulating factors, monoclonal antibodies, vaccines, gene therapy, and nonspecific immunomodulating agents.

Interferons (IFNs) are types of cytokines that occur naturally in the body. Interferon alpha, interferon beta, and interferon gamma are examples of interferons that may be used in cancer treatment.

Like interferons, interleukins (ILs) are cytokines that occur naturally in the body and can be made in the laboratory. Many interleukins have been identified for the treatment of cancer. For example, interleukin-2 (IL-2 or aldesleukin), interleukin 7, and interleukin 12 have may be used as an anti-cancer treatment. IL-2 may stimulate the growth and activity of many immune cells, such as lymphocytes, that can destroy cancer cells. Interleukins may be used to treat a number of cancers, including leukemia, lymphoma, and brain, colorectal, ovarian, breast, kidney and prostate cancers.

Colony-stimulating factors (CSFs) (sometimes called hematopoietic growth factors) may also be used for the treatment of cancer. Some examples of CSFs include, but are not limited to, G-CSF (filgrastim) and GM-CSF (sargramostim). CSFs may promote the division of bone marrow stem cells and their development into white blood cells, platelets, and red blood cells. Bone marrow is critical to the body's immune system because it is the source of all blood cells. Because anticancer drugs can damage the body's ability to make white blood cells, red blood cells, and platelets, stimulation of the immune system by CSFs may benefit subjects undergoing other anti-cancer treatment, thus CSFs may be combined with other anti-cancer therapies, such as chemotherapy. CSFs may be used to treat a large variety of cancers, including lymphoma, leukemia, multiple myeloma, melanoma, and cancers of the brain, lung, esophagus, breast, uterus, ovary, prostate, kidney, colon, and rectum.

Another type of biological therapy includes monoclonal antibodies (MOABs or MoABs). These antibodies may be produced by a single type of cell and may be specific for a particular antigen. To create MOABs, a human cancer cells may be injected into mice. In response, the mouse immune system can make antibodies against these cancer cells. The mouse plasma cells that produce antibodies may be isolated and fused with laboratory-grown cells to create “hybrid” cells called hybridomas. Hybridomas can indefinitely produce large quantities of these pure antibodies, or MOABs. MOABs may be used in cancer treatment in a number of ways. For instance, MOABs that react with specific types of cancer may enhance a subject's immune response to the cancer. MOABs can be programmed to act against cell growth factors, thus interfering with the growth of cancer cells.

MOABs may be linked to other anti-cancer therapies such as chemotherapeutics, radioisotopes (radioactive substances), other biological therapies, or other toxins. When the antibodies latch onto cancer cells, they deliver these anti-cancer therapies directly to the tumor, helping to destroy it. MOABs carrying radioisotopes may also prove useful in diagnosing certain cancers, such as colorectal, ovarian, prostate and breast.

Rituxan® (rituximab) and Herceptin® (trastuzumab) are examples of MOABs that may be used as a biological therapy. Rituxan may be used for the treatment of non-Hodgkin lymphoma. Herceptin can be used to treat metastatic breast cancer in subjects with tumors that produce excess amounts of a protein called HER2. Alternatively, MOABs may be used to treat lymphoma, leukemia, melanoma, and cancers of the brain, breast, lung, kidney, colon, rectum, ovary, prostate, and other areas.

Cancer vaccines are another form of biological therapy. Cancer vaccines may be designed to encourage the subject's immune system to recognize cancer cells. Cancer vaccines may be designed to treat existing cancers (therapeutic vaccines) or to prevent the development of cancer (prophylactic vaccines). Therapeutic vaccines may be injected in a person after cancer is diagnosed. These vaccines may stop the growth of existing tumors, prevent cancer from recurring, or eliminate cancer cells not killed by prior treatments. Cancer vaccines given when the tumor is small may be able to eradicate the cancer. On the other hand, prophylactic vaccines are given to healthy individuals before cancer develops. These vaccines are designed to stimulate the immune system to attack viruses that can cause cancer. By targeting these cancer-causing viruses, development of certain cancers may be prevented. For example, cervarix and gardasil are vaccines to treat human papilloma virus and may prevent cervical cancer. Therapeutic vaccines may be used to treat melanoma, lymphoma, leukemia, and cancers of the brain, breast, lung, kidney, ovary, prostate, pancreas, colon, and rectum. Cancer vaccines can be used in combination with other anti-cancer therapies.

Gene therapy is another example of a biological therapy. Gene therapy may involve introducing genetic material into a person's cells to fight disease. Gene therapy methods may improve a subject's immune response to cancer. For example, a gene may be inserted into an immune cell to enhance its ability to recognize and attack cancer cells. In another approach, cancer cells may be injected with genes that cause the cancer cells to produce cytokines and stimulate the immune system.

In some instances, biological therapy includes nonspecific immunomodulating agents. Nonspecific immunomodulating agents are substances that stimulate or indirectly augment the immune system. Often, these agents target key immune system cells and may cause secondary responses such as increased production of cytokines and immunoglobulins. Two nonspecific immunomodulating agents used in cancer treatment are bacillus Calmette-Guerin (BCG) and levamisole. BCG may be used in the treatment of superficial bladder cancer following surgery. BCG may work by stimulating an inflammatory, and possibly an immune, response. A solution of BCG may be instilled in the bladder. Levamisole is sometimes used along with fluorouracil (5-FU) chemotherapy in the treatment of stage III (Dukes' C) colon cancer following surgery. Levamisole may act to restore depressed immune function.

Photodynamic therapy (PDT) is an anti-cancer treatment that may use a drug, called a photosensitizer or photosensitizing agent, and a particular type of light. When photosensitizers are exposed to a specific wavelength of light, they may produce a form of oxygen that kills nearby cells. A photosensitizer may be activated by light of a specific wavelength. This wavelength determines how far the light can travel into the body. Thus, photosensitizers and wavelengths of light may be used to treat different areas of the body with PDT.

In the first step of PDT for cancer treatment, a photosensitizing agent may be injected into the bloodstream. The agent may be absorbed by cells all over the body but may stay in cancer cells longer than it does in normal cells. Approximately 24 to 72 hours after injection, when most of the agent has left normal cells but remains in cancer cells, the tumor can be exposed to light. The photosensitizer in the tumor can absorb the light and produces an active form of oxygen that destroys nearby cancer cells. In addition to directly killing cancer cells, PDT may shrink or destroy tumors in two other ways. The photosensitizer can damage blood vessels in the tumor, thereby preventing the cancer from receiving necessary nutrients. PDT may also activate the immune system to attack the tumor cells.

The light used for PDT can come from a laser or other sources. Laser light can be directed through fiber optic cables (thin fibers that transmit light) to deliver light to areas inside the body. For example, a fiber optic cable can be inserted through an endoscope (a thin, lighted tube used to look at tissues inside the body) into the lungs or esophagus to treat cancer in these organs. Other light sources include light-emitting diodes (LEDs), which may be used for surface tumors, such as skin cancer. PDT is usually performed as an outsubject procedure. PDT may also be repeated and may be used with other therapies, such as surgery, radiation, or chemotherapy.

Extracorporeal photopheresis (ECP) is a type of PDT in which a machine may be used to collect the subject's blood cells. The subject's blood cells may be treated outside the body with a photosensitizing agent, exposed to light, and then returned to the subject. ECP may be used to help lessen the severity of skin symptoms of cutaneous T-cell lymphoma that has not responded to other therapies. ECP may be used to treat other blood cancers, and may also help reduce rejection after transplants.

Additionally, photosensitizing agent, such as porfimer sodium or Photofrin®, may be used in PDT to treat or relieve the symptoms of esophageal cancer and non-small cell lung cancer. Porfimer sodium may relieve symptoms of esophageal cancer when the cancer obstructs the esophagus or when the cancer cannot be satisfactorily treated with laser therapy alone. Porfimer sodium may be used to treat non-small cell lung cancer in subjects for whom the usual treatments are not appropriate, and to relieve symptoms in subjects with non-small cell lung cancer that obstructs the airways. Porfimer sodium may also be used for the treatment of precancerous lesions in subjects with Barrett esophagus, a condition that can lead to esophageal cancer.

Laser therapy may use high-intensity light to treat cancer and other illnesses. Lasers can be used to shrink or destroy tumors or precancerous growths. Lasers are most commonly used to treat superficial cancers (cancers on the surface of the body or the lining of internal organs) such as basal cell skin cancer and the very early stages of some cancers, such as cervical, penile, vaginal, vulvar, and non-small cell lung cancer.

Lasers may also be used to relieve certain symptoms of cancer, such as bleeding or obstruction. For example, lasers can be used to shrink or destroy a tumor that is blocking a subject's trachea (windpipe) or esophagus. Lasers also can be used to remove colon polyps or tumors that are blocking the colon or stomach.

Laser therapy is often given through a flexible endoscope (a thin, lighted tube used to look at tissues inside the body). The endoscope is fitted with optical fibers (thin fibers that transmit light). It is inserted through an opening in the body, such as the mouth, nose, anus, or vagina. Laser light is then precisely aimed to cut or destroy a tumor.

Laser-induced interstitial thermotherapy (LITT), or interstitial laser photocoagulation, also uses lasers to treat some cancers. LITT is similar to a cancer treatment called hyperthermia, which uses heat to shrink tumors by damaging or killing cancer cells. During LITT, an optical fiber is inserted into a tumor. Laser light at the tip of the fiber raises the temperature of the tumor cells and damages or destroys them. LITT is sometimes used to shrink tumors in the liver.

Laser therapy can be used alone, but most often it is combined with other treatments, such as surgery, chemotherapy, or radiation therapy. In addition, lasers can seal nerve endings to reduce pain after surgery and seal lymph vessels to reduce swelling and limit the spread of tumor cells.

Lasers used to treat cancer may include carbon dioxide (CO₂) lasers, argon lasers, and neodymium:yttrium-aluminum-garnet (Nd:YAG) lasers. Each of these can shrink or destroy tumors and can be used with endoscopes. CO₂ and argon lasers can cut the skin's surface without going into deeper layers. Thus, they can be used to remove superficial cancers, such as skin cancer. In contrast, the Nd:YAG laser is more commonly applied through an endoscope to treat internal organs, such as the uterus, esophagus, and colon. Nd:YAG laser light can also travel through optical fibers into specific areas of the body during LITT. Argon lasers are often used to activate the drugs used in PDT.

For subjects with systemic disease, additional treatment modalities such as adjuvant chemotherapy (e.g., docetaxel, mitoxantrone and prednisone) and systemic radiation therapy (e.g., samarium or strontium) can be designated. Such subjects would likely be treated immediately with radiation therapy in order to eliminate presumed micro-metastatic disease, which cannot be detected clinically but can be revealed by the target gene expression signature. Such subjects can also be more closely monitored for signs of disease progression.

For subjects that do not have systemic disease, localized adjuvant radiation therapy (e.g., localized to breast tissue) or endocrine therapy or chemotherapy may be administered. For subjects with no evidence of disease (NED), expression profiling and/or calculation of a risk score, as described herein, may be used to determine the risk of recurrence of the breast cancer. For subjects identified as having a low risk of recurrence of breast cancer and identified as having a high benefit of radiotherapy using the methods described herein, adjuvant radiation therapy would be recommended.

Target genes can be grouped so that information obtained about the set of target genes in the group can be used to make or assist in making a clinically relevant judgment such as a diagnosis, prognosis, or treatment choice.

A subject report is also provided comprising a representation of measured expression levels of a plurality of target genes in a biological sample from the subject, wherein the representation comprises expression levels of target genes corresponding to any one, two, three, four, five, six, eight, ten, twenty, thirty or more of the target genes, the subsets described herein, or a combination thereof. In some embodiments, the representation of the measured expression level(s) may take the form of a linear or nonlinear combination of expression levels of the target genes of interest. The subject report may be provided in a machine (e.g., a computer) readable format and/or in a hard (paper) copy. The report can also include standard measurements of expression levels of said plurality of target genes from one or more sets of subjects with known disease status and/or outcome. The report can be used to inform the subject and/or treating physician of the expression levels of the expressed target genes, the likely medical diagnosis and/or implications, and optionally may recommend a treatment modality for the subject.

Also provided are representations of the gene expression profiles useful for treating, diagnosing, prognosticating, and otherwise assessing disease. In some embodiments, these profile representations are reduced to a medium that can be automatically read by a machine such as computer readable media (magnetic, optical, and the like). The articles can also include instructions for assessing the gene expression profiles in such media. For example, the articles may comprise a readable storage form having computer instructions for comparing gene expression profiles of the portfolios of genes described above. The articles may also have gene expression profiles digitally recorded therein so that they may be compared with gene expression data from subject samples. Alternatively, the profiles can be recorded in different representational format. A graphical recordation is one such format. Clustering algorithms can assist in the visualization of such data.

### EXPERIMENTAL

The following example is provided in order to demonstrate and further illustrate certain preferred embodiments and aspects of the present invention and are not to be construed as limiting the scope thereof. Use of pronouns such as, “we”, “our,” and “I” refer to the inventive entity.

### Example 1: Development and Validation of a Genomic Classifier for Prognosis of Local Recurrence of Breast Cancer and Prediction of Response to Radiation Therapy

### Methods

### SweBCG91-RT Cohort

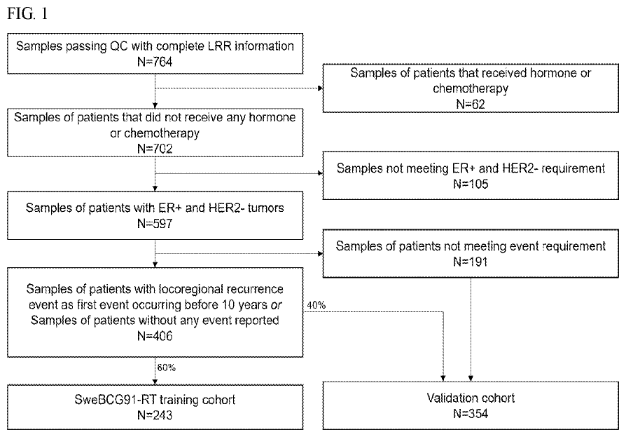

SweBCG91-RT was a randomized trial conducted in Sweden and has been described previously (see, Malmström, P. et al. European journal of cancer (Oxford, England: 1990) 39, 1690-1697, doi:10.1016/s0959-8049(03)00324-1 (2003); Killander, F. et al. European journal of cancer (Oxford, England: 1990) 67, 57-65, (2016); Sjöström, M. et al. Journal of Clinical Oncology 37, 3340-3349 (2019)). Briefly, the trial randomized 1,178 patients with node-negative, stage I-IIA breast cancer to adjuvant whole-breast RT or no RT following BCS. All patients had negative surgical margins. Endocrine therapy and chemotherapy were provided according to local guidelines at the time and was only administered to 8% of patients in the original trial. Subtyping by immunohistochemical staining of ER, PgR, HER2 and KI67 was performed as previously described (see, Sjöström, M. et al. Journal of Clinical Oncology 35, 3222-3229 (2017)). 597 samples from patients with ER+, HER2− tumors had available gene expression data and complete LRR information. This patient subset was further divided into a training cohort (N=243) and a validation cohort (N=354) (Additional information below and FIG. 1).

### Princess Margaret Cohort

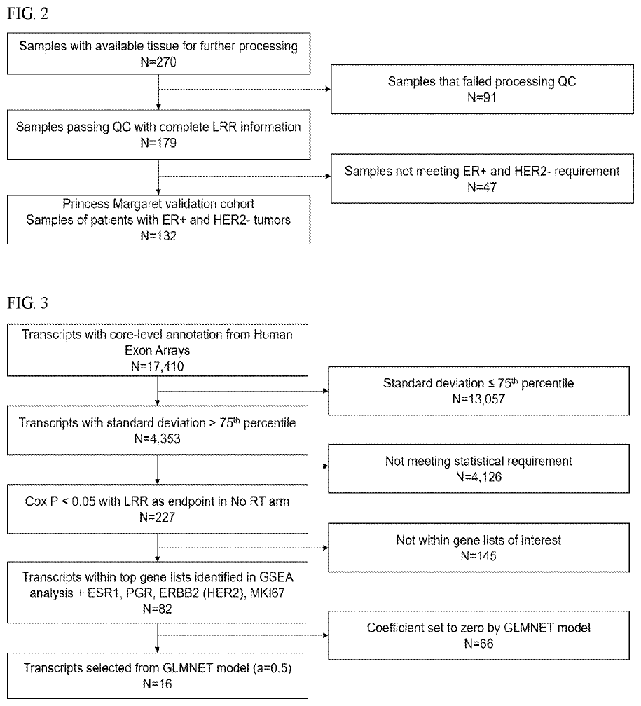

The Princess Margaret cohort was a randomized trial conducted in Canada and has been previously described (see, Fyles, A. W. et al. The New England journal of medicine 351, 963-970 (2004); Liu, F. F. et al. Journal of clinical oncology: official journal of the American Society of Clinical Oncology 33, 2035-2040 (2015)). Briefly, the trial randomized 769 women age 50 years or older with T1 or T2 node-negative breast cancer to adjuvant whole-breast RT or not RT following BCS. All patients in the Princess Margaret trial were treated with tamoxifen. Subtyping by immunohistochemical staining of ER, PgR, HER2 and Ki67 was performed as previously described (see, Liu, F. F. et al. Journal of clinical oncology: official journal of the American Society of Clinical Oncology 33, 2035-2040 (2015)). Gene expression data was available from 132 samples from patients with ER+, HER2− tumors and complete LRR information (FIG. 2).

### Gene Expression Data

Gene expression data (Gene Expression Omnibus, GSE119295) were obtained from primary tumors of 764 patients included in the SweBCG91-RT trial (see, Sjöström, M. et al. Journal of Clinical Oncology 37, 3340-3349 (2019)). Similar to the SweBCG91-RT cohort, gene expression data for the Princess Margaret cohort were generated from GeneChip Human Exon 1.0 ST Arrays (Thermo Fisher Scientific, South San Francisco) in a CLIA/CAP-certified laboratory (Decipher Biosciences, San Diego, CA).

### Development of Training and Validation Sets

In this analysis, only patients with ER+, HER2− tumors were included. This was to ensure that the resulting signature could distinguish risk among clinically low-risk patients who would be candidates for RT omission. Furthermore, as systemic therapy can influence LRR and only a small portion of SweBCG91-RT patients were treated systemically, patients treated with chemotherapy and/or endocrine therapy were removed from analysis of the SweBCG91-RT cohort to improve result interpretation. After these considerations, 597 patients of the SweBCG91-RT trial were included in this study.

To improve the signal to noise ratio for identification of a LRR event in the training cohort comprised of SweBCG91-RT data, only patients with a recorded locoregional event as first event prior to 10 years or patients without any recorded event with a follow-up time greater than 10 years were included. 60% of these patients were retained for the training cohort and all remaining patients used for validation (FIG. 1). There were no significant differences in the distribution of clinicopathologic variables between the No RT and RT arms of the SweBCG91-RT training and validation cohorts (Tables 1 and 2), with the exception of age in the training cohort, where there was an increased proportion of women aged 40-59 in the RT arm relative to the no RT arm. The lower rate of ipsilateral breast tumor recurrence in the RT arm compared to No RT was preserved in both the training and validation cohorts (Table 3). The entirety of the Princess Margaret cohort with available genomic and LRR data from patients with ER+, HER2− tumors was reserved for validation and none were used for training purposes.

### Gene Set Enrichment Analysis

To identify gene sets related to LRR, Gene Set Enrichment Analysis (GSEA) was performed within the 131 patients of the training cohort not treated with RT (see, Subramanian, A. et al. Proc Natl Acad Sci U S A 102, 15545-15550, (2005); Mootha, V. K. et al. Nat Genet 34, 267-273 (2003)). First, for each annotated gene transcript, a Cox model to LRR was calculated. A pre-ranked gene list was then entered into GSEA, where for each gene, a statistic was created with the following equation: −log (p-value)*(if HR>1,1, else −1), where p-value and hazard ratio (HR) are from Cox model to LRR (see, Table 8 for the determined statistic/coefficient values for each of the genes). Thus, individual genes highly prognostic for higher LRR rate in patients will have large positive values, and individual genes highly prognostic for lower LRR rate in patients will have large negative values. The ranked list was entered into GSEA (version 4.0.3) and the Hallmarks (H), C2, and C5 collections were specified (version 7.2 of Molecular Signatures database, MSigDB) (see, Liberzon, A. et al. Cell systems 1, 417-425 (2015)). Due to inherent differences among gene set collections, the H, C2, and C5 lists were analyzed separately to look for trends across collections. For the genes analyzed, a total enrichment score was cumulated based on the coefficient values determined for each gene (see, Table 8). Gene sets with positive enrichment scores were interpreted as associated with worse prognosis (increase of LRRs), and gene sets with negative enrichment scores were interpreted as associated with better prognosis (decrease of LRRs).

### Model Development

Genes were identified as potential candidates for the model from the GSEA gene lists with a false discovery rate q-value <0.05 and in the top ten of positive and negative gene lists of the H, C2, and C5 collections (Table 4). These nominated genes, along with the genes encoding for ER, PgR, HER2 and Ki67, were intersected with genes that met the following requirements: genes with a standard deviation in the top quartile of standard deviation values and genes with a p-value <0.05 when evaluated in a Cox model to the LRR endpoint in patients not treated with RT in the training set (FIG. 3). The resulting 82 genes were fed into an elastic net regression model (R glmnet, alpha=0.5) for training in the training cohort, among patients not treated with RT, using LRR as the endpoint. Elastic net regression modeling resulted in a final set of 16 genes for the model. The scores were dichotomized to high vs low by applying a pre-specified cut-off using the 25^{th} percentile value of the scores in the SweBCG91-RT training dataset. This threshold was then applied to the SweBCG91-RT and Princess Margaret validation sets.

### Statistical Methods

Statistical analyses were performed using R, version 4.0.2 (https://cran.r-project.org/bin/windows/base/old/4.0.2/). The primary end point was cumulative incidence of LRR using time to LRR as first event. Cumulative incidences were computed using a competing risk approach (R cmprsk package) and HRs and tests for significant differences between the dichotomized risk groups were calculated using cause-specific Cox proportional hazards regression. As previously described, the hazards are not proportional over time for several variables in the SweBCG91RT cohort when checked graphically and with Schoenfeld's test, and the HRs should be interpreted as the mean over the entire time period (see, Liberzon, A. et al. Cell systems 1, 417-425 (2015)). Distant metastasis and death without recurrence were considered competing events. Patients with synchronous distant metastasis and LRR, defined as LRR registered at the same time or within 3 months as the metastasis were regarded as having a LRR. Hazard ratios and point estimates are reported with 95% confidence intervals (CI) in brackets. A p-value less than 0.05 was considered statistically significant. For demographic tables, differences in group distributions between the treated and untreated arms were tested using the Chi-squared test with Yates' continuity correction for 2×2 tables. The univariable and multivariable Cox analyses, and the test for interaction was performed using continuous signature scores, thus, not dependent on any threshold. The test for interaction between score and RT included all patients in the SweBCG91-RT and Princess Margaret validation cohorts and was stratified by cohort.

### Results

### Biological Concepts Associated With Locoregional Recurrence

In patients not treated with RT in the SweBCG91-RT training cohort, we used GSEA to identify biological concepts associated with LRR in breast cancer. The top positively enriched gene lists across the Molecular Signatures database Hallmark, C2 and C5 collections included lists involving cell cycle and proliferation (Table 4), whereas top negatively enriched gene lists included lists related to the immune system.

Top gene lists were intersected with the most informative and individually prognostic genes for LRR in the patients of the training set that were not given RT. A generalized linear model with elastic net regularization was trained using the LRR endpoint, and the resulting 16-gene signature includes genes that incorporate the concepts that are broadly shown from the GSEA lists (Table 5). We named this signature the Profile for the Omission of Local Adjuvant Radiation (POLAR). Table 8 shows the determined statistic/coefficient values for each of the genes utilized in generating an enrichment score for assessing prognosis for LRR in patients.

A linear algorithm was used to generate a risk score and categorize patients as low risk or high risk using POLAR. Specifically, expression levels for each of the 16 genes in the signature (Table 5) were determined and multiplied by their respective coefficient values (Table 8) to produce a weighted expression value for each gene assayed. The weighted expression values were then added together to generate a final risk score. A pre-determined threshold of 0.5622 was then applied to the generated risk scores to assign patients to high or low risk categories. For instance, patients with a risk score higher than 0.5622 were categorized as high risk and patients with a risk score lower than 0.5622 were categorized as low risk.

### POLAR is Prognostic for LRR in Patients Not Treated With RT in the SweBCG91-RT Validation Cohort

In the SweBCG91-RT validation cohort, POLAR was prognostic for LRR in patients not treated with radiotherapy in a univariable Cox model with LRR as the endpoint (HR=1.7[1.3,2.2], p<0.001). The signature remained prognostic in a multivariable model including age, grade, tumor size, and luminal A vs luminal B (HR=1.7 [1.2,2.3], p<0.001) (Table 6).

### Differential Effect of RT on Patients With Low and High POLAR Risk in SweBCG91-RT and Princess Margaret Validation Cohorts

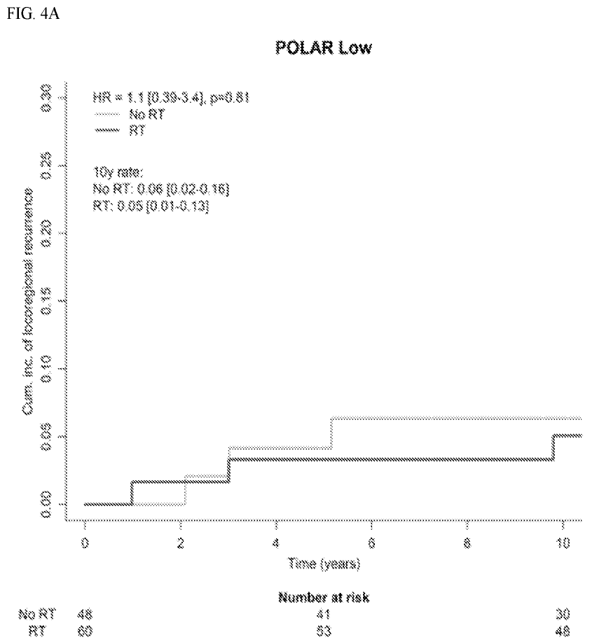

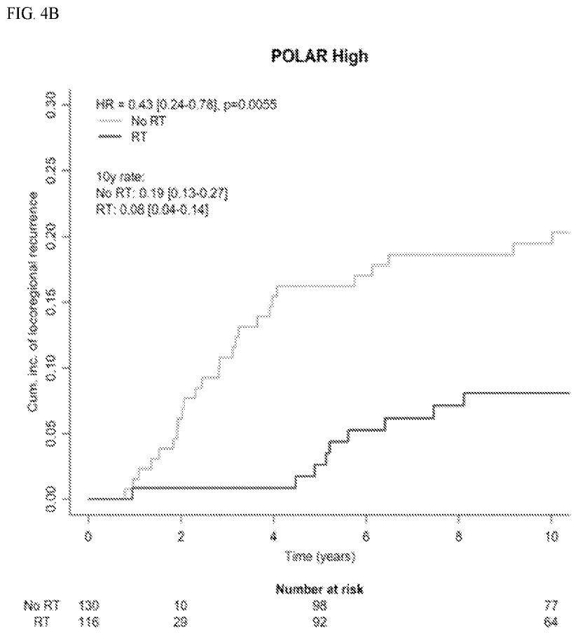

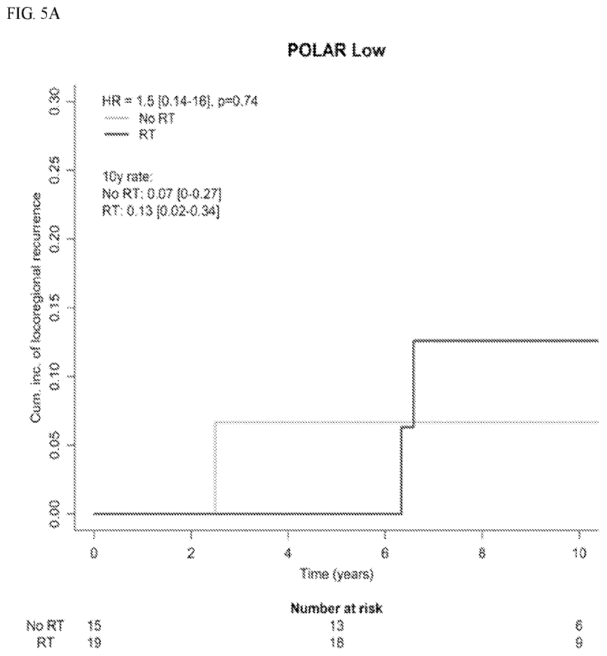

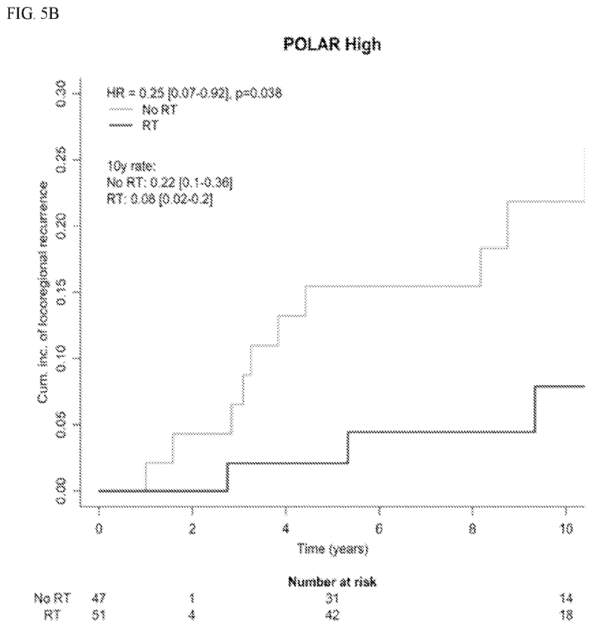

The cumulative incidence of LRR for SweBCG91-RT patients categorized as low risk in the validation cohort by POLAR was less than 10% at 10-years regardless if they received radiotherapy or not and a benefit from RT could not be shown (10-year LRR cumulative incidence No RT: 6% [2%-16%], RT: 5% [1%-13%], HR=1.1 [0.39-3.4], p=0.81, FIG. 4A). However, these results must be interpreted with caution given the small number of patients (N=108). For SweBCG91-RT patients categorized as high-risk by the signature, RT resulted in a significantly lower risk of LRR compared to those who did not receive RT (10-year locoregional cumulative incidence for No RT: 19% [13%-27%], RT: 8% [4%-14%], HR=0.43 [0.24-0.78], p=0.0055, FIG. 4B). We then evaluated POLAR in 132 patients of the Princess Margaret trial (FIG. 5). Demographic and descriptive details for this cohort are provided in Table 7. For women treated with tamoxifen alone after BCS, patients categorized as low risk by POLAR had a 10-year cumulative incidence of LRR of 7% [0%-27%] without RT and 13% [2%-34%] with RT. A benefit from RT could not be shown in these low risk women (HR=1.5 [0.14-16], p=0.74), although the small number of patients (N=34) preclude a definitive conclusion (FIG. 5A). Women classified by POLAR as high risk had a cumulative incidence of LRR at 10 years of 22% [10%-36%] without RT, and 8% [2%-20%] with RT, and a significant benefit of RT (HR=0.25 [0.07-0.92], p=0.038). We performed an exploratory analysis by testing for interaction between RT and the continuous POLAR score in the total of N=486 patients of the SweBCG91-RT and Princess Margaret evaluable samples and the interaction p-value was 0.066.

These results showed that methods and classifiers of the present disclosure are prognostic for locoregional recurrence and identify breast cancer patients who would not benefit from adjuvant radiotherapy (radiation treatment). The results also showed that the methods and classifiers of the disclosure can identify breast cancer patients who have a low risk of locoregional recurrence. These results further showed that the methods and classifiers of the present are useful for treating breast cancer.

The genomic classifier of the present disclosure identified patients at low risk of LRR without adjuvant radiotherapy post-breast conserving surgery, thus identifying them as candidates for omission of radiotherapy after breast conserving surgery. Further, the genomic classifier of the present disclosure showed that breast cancer patients of lowest risk of LRR have no significant benefit from RT.

TABLE 1
SweBCG91-RT training cohort demographics

| | No RT | RT | Total | p- |
|---|---|---|---|---|
| N (%) | N = 131 | N = 112 | N = 243 | value |
| Age at surgery | | | | |
| (years) | | | | |
| ≤39 | 5 (3.8) | 1 (0.9) | 6 (2.5) | 0.03 |
| 40-49 | 26 (19.8) | 26 (23.2) | 52 (21.4) | |
| 50-59 | 34 (26) | 47 (42) | 81 (33.3) | |
| 60-69 | 57 (43.5) | 34 (30.4) | 91 (37.4) | |
| ≥70 | 9 (6.9) | 4 (3.6) | 13 (5.3) | |
| Menopausal status | | | | |
| Premenopausal | 33 (25.2) | 24 (21.4) | 57 (23.5) | 0.63 |
| Postmenopausal | 95 (72.5) | 84 (75) | 179 (73.7) | |
| NA | 3 (2.3) | 4 (3.6) | 7 (2.9) | |
| Histologic grade | | | | |
| Grade 1 | 14 (10.7) | 24 (21.4) | 38 (15.6) | 0.07 |
| Grade 2 | 99 (75.6) | 73 (65.2) | 172 (70.8) | |
| Grade 3 | 18 (13.7) | 14 (12.5) | 32 (13.2) | |
| NA | 0 (0) | 1 (0.9) | 1 (0.4) | |
| Tumor size (mm) | | | | |
| ≤10 | 55 (42) | 43 (38.4) | 98 (40.3) | 0.75 |
| 11-20 | 71 (54.2) | 64 (57.1) | 135 (55.6) | |
| 21-30 | 4 (3.1) | 3 (2.7) | 7 (2.9) | |
| NA | 1 (0.8) | 2 (1.8) | 3 (1.2) | |
| Subtype by IHC | | | | |
| Luminal A | 93 (71) | 73 (65.2) | 166 (68.3) | 0.41 |
| Luminal B (HER2−) | 38 (29) | 39 (34.8) | 77 (31.7) | |
| Adjuvant endocrine | | | | |
| therapy | | | | |
| No | 131 (100) | 112 (100) | 243 (100) | |
| Adjuvant | | | | |
| chemotherapy | | | | |
| No | 131 (100) | 112 (100) | 243 (100) | |

TABLE 2
SweBCG91-RT validation cohort demographics

| | No RT | RT | Total | p- |
|---|---|---|---|---|
| N (%) | N = 178 | N = 176 | N = 354 | value |
| Age at surgery | | | | |
| (years) | | | | |
| ≤39 | 4 (2.2) | 3 (1.7) | 7 (2) | 0.69 |
| 40-49 | 34 (19.1) | 26 (14.8) | 60 (16.9) | |
| 50-59 | 54 (30.3) | 49 (27.8) | 103 (29.1) | |
| 60-69 | 56 (31.5) | 64 (36.4) | 120 (33.9) | |
| ≥70 | 30 (16.9) | 34 (19.3) | 64 (18.1) | |
| Menopausal status | | | | |
| Premenopausal | 36 (20.2) | 29 (16.5) | 65 (18.4) | 0.37 |
| Postmenopausal | 135 (75.8) | 145 (82.4) | 280 (79.1) | |
| NA | 7 (3.9) | 2 (1.1) | \|9 (2.5) | |
| Histologic grade | | | | |
| Grade 1 | 32 (18) | 28 (15.9) | 60 (16.9) | 0.36 |
| Grade 2 | 108 (60.7) | 118 (67) | 226 (63.8) | |
| Grade 3 | 34 (19.1) | 25 (14.2) | 59 (16.7) | |
| NA | 4 (2.2) | 5 (2.8) | 9 (2.5) | |
| Tumor size (mm) | | | | |
| ≤10 | 70 (39.3) | 69 (39.2) | 139 (39.3) | 0.78 |
| 11-20 | 97 (54.5) | 99 (56.2) | 196 (55.4) | |
| 21-30 | 11 (6.2) | 8 (4.5) | 19 (5.4) | |
| Subtype by IHC | | | | |
| Luminal A | 115 (64.6) | 113 (64.2) | 228 (64.4) | 1.00 |
| Luminal B (HER2−) | 63 (35.4) | 63 (35.8) | 126 (35.6) | |
| Adjuvant endocrine | | | | |
| therapy | | | | |
| No | 178 (100) | 176 (100) | 354 (100) | |
| Adjuvant | | | | |
| chemotherapy | | | | |
| No | 178 (100) | 176 (100) | 354 (100) | |

TABLE 3
Distribution of events in SweBCG91-RT training and validation cohorts
Training cohort (N = 243)

| | No RT | RT | | p- |
|---|---|---|---|---|
| N (%) | (N = 131) | (N = 112) | Total | value |
| Ipsilateral breast | | | | |
| tumor recurrence | | | | |
| No | 91 (69.5) | 97 (86.6) | 188 (77.4) | 0.0025 |
| Yes | 40 (30.5) | 15 (13.4) | 55 (22.6) | |
| Regional recurrence | | | | |
| No | 125 (95.4) | 109 (97.3) | 234 (96.3) | 0.66 |
| Yes | 6 (4.6) | 3 (2.7) | 9 (3.7) | |
| Distant metastasis | | | | |
| No | 120 (91.6) | 107 (95.5) | 227 (93.4) | 0.33 |
| Yes | 11 (8.4) | 5 (4.5) | 16 (6.6) | |
| Breast cancer specific | | | | |
| death | | | | |
| No | 116 (88.5) | 102 (91.1) | 218 (89.7) | 0.66 |
| Yes | 15 (11.5) | 10 (8.9) | 25 (10.3) | |
| Death from any cause | | | | |
| No | 89 (67.9) | 84 (75) | 173 (71.2) | 0.28 |
| Yes | 42 (32.1) | 28 (25) | 70 (28.8) | |

Validation cohort (N = 354)

| | No RT | RT | Total | p- |
|---|---|---|---|---|
| | (N = 178) | (N = 176) | (N = 354) | value |
| Ipsilateral breast | | | | |
| tumor recurrence | | | | |
| No | 135 (75.8) | 153 (86.9) | 288 (81.4) | 0.01 |
| Yes | 43 (24.2) | 23 (13.1) | 66 (18.6) | |
| Regional recurrence | | | | |
| No | 170 (95.5) | 172 (97.7) | 342 (96.6) | 0.39 |
| Yes | 8 (4.5) | 4 (2.3) | 12 (3.4) | |
| Distant metastasis | | | | |
| No | 151 (84.8) | 146 (83) | 297 (83.9) | 0.74 |
| Yes | 27 (15.2) | 30 (17) | 57 (16.1) | |
| Breast cancer specific | | | | |
| death | | | | |
| No | 141 (79.2) | 136 (77.3) | 277 (78.2) | 0.75 |
| Yes | 37 (20.8) | 40 (22.7) | 77 (21.8) | |
| Death from any cause | | | | |
| No | 79 (44.4) | 77 (43.8) | 156 (44.1) | 0.99 |
| Yes | 99 (55.6) | 99 (56.2) | 198 (55.9) | |

TABLE 4
Gene lists identified by GSEA as highly positively and negatively enriched.

| | Positive | Negative |
|---|---|---|
| | (associated with worse prognosis) | (associated with better prognosis) |
| H | HALLMARK_E2F_TARGETS | HALLMARK_TNFA_SIGNALING_ |
| | HALLMARK_MYC_TARGETS_V2 | VIA_NFKB |
| | HALLMARK_G2M_CHECKPOINT | HALLMARK_KRAS_SIGNALING_ |
| | HALLMARK_MYC_TARGETS_V1 | UP |
| | HALLMARK_ESTROGEN_RESPONSE_LATE | HALLMARK_ALLOGRAFT_REJECTION |
| | HALLMARK_MYOGENESIS | |
| | HALLMARK_OXIDATIVE_PHOSPHORYLATION | |
| | HALLMARK_ESTROGEN_RESPONSE_EARLY | |
| | HALLMARK_MTORCI_SIGNALING | |
| | HALLMARK_HEDGEHOG_SIGNALING | |
| C2 | SOTIRIOU_BREAST_CANCER_GRADE_1_ | SMID_BREAST_CANCER_NORMAL_ |
| | VS_3_UP | LIKE_UP |
| | ROSTY_CERVICAL_CANCER_PROLIFERATION_ | WINTER_HYPOXIA_DN |
| | CLUSTER | PHONG_TNF_TARGETS_UP |
| | NIKOLSKY_BREAST_CANCER_16P13_ | ZHENG_FOXP3_TARGETS_IN_T_ |
| | AMPLICON | LYMPHOCYTE_DN |
| | ZHOU_CELL_CYCLE_GENES_IN_IR_RESPONSE_ | KEGG_GRAFT_VERSUS_HOST_ |
| | 24HR | DISEASE |
| | BLANCO_MELO_BRONCHIAL_EPITHELIAL_ | BOQUEST_STEM_CELL_CULTURED_ |
| | CELLS_INFLUENZA_A_DEL_ | VS_FRESH_DN |
| | NS1_INFECTION_DN | BERTUCCI_INVASIVE_CARCINOMA_ |
| | DUTERTRE_ESTRADIOL_RESPONSE_24HR_UP | DUCTAL_VS_LOBULAR_DN |
| | WHITEFORD_PEDIATRIC_CANCER_ | NAKAYAMA_SOFT_TISSUE_TUMORS_ |
| | MARKERS | PCA2_DN |
| | ZHAN_MULTIPLE_MYELOMA_PR_UP | REACTOME_CHEMOKINE_RECEPTORS_ |
| | LEE_EARLY_T_LYMPHOCYTE_UP | BIND_CHEMOKINES |
| | CHIANG_LIVER_CANCER_SUBCLASS_ | KEGG_AUTOIMMUNE_THYROID_ |
| | PROLIFERATION_UP | DISEASE |
| C5 | GO_ANAPHASE_PROMOTING_COMPLEX_ | GO_B_CELL_RECEPTOR_SIGNALING_ |
| | DEPENDENT_CATABOLIC_PROCESS | PATHWAY |
| | GO_DNA_REPLICATION_INDEPENDENT_ | GO_POSITIVE_REGULATION_OF_ |
| | NUCLEOSOME_ORGANIZATION | INTERLEUKIN_2_PRODUCTION |
| | GO_CENTROMERE_COMPLEX_ASSEMBLY | GO_INTERLEUKIN_1_BETA_PRODUCTION |
| | GO_CHROMATIN_REMODELING_AT_ | GO_REGULATION_OF_ANTIGEN_ |
| | CENTROMERE | RECEPTOR_MEDIATED_SIGNALING_ |
| | | PATHWAY |
| | | GO_NEGATIVE_REGULATION_OF_ |
| | | SMOOTH_MUSCLE_CELL_PROLIFERATION |
| | | GO_INTERLEUKIN_1_PRODUCTION |
| | | GO_REGULATION_OF_B_CELL_ |
| | | RECEPTOR_SIGNALING_PATHWAY |
| | | GO_RESPONSE_TO_CHEMOKINE |
| | | GO_LYMPHOCYTE_CHEMOTAXIS |
| | | GO_DENDRITIC_CELL_CHEMOTAXIS |

TABLE 5
16 genes in the model

| | |
|---|---|
| AGR2 | Anterior Gradient 2, Protein Disulphide Isomerase |
| B4GALT1 | Beta-1,4-Galactosyltransferase 1 |
| CLDN7 | Claudin 7 |
| EZR | Ezrin |
| GNG11 | G Protein Subunit Gamma 11 |
| JUN | Jun Proto-Oncogene |
| MMP11 | Matrix Metallopeptidase 11 |
| PKIB | CAMP-Dependent Protein Kinase Inhibitor Beta |
| PRPS1 | Phosphoribosyl Pyrophosphate Synthetase 1 |
| PSMD10 | Proteasome 26S Subunit, Non-ATPase 10 |
| SH3BP5 | SH3 Domain Binding Protein 5 |
| SLC16A3 | Solute Carrier Family 16 Member 3 |
| SLC7A11 | Solute Carrier Family 7 Member 11 |
| SPP1 | Secreted Phosphoprotein 1 |
| TNNT1 | Troponin T1, Slow Skeletal Type |
| UBE2E1 | Ubiquitin Conjugating Enzyme E2 E1 |

TABLE 6

| Univariable analysis | | HR [95% CI] | p-value |
|---|---|---|---|
| POLAR | | 1.7 [1.3,2.2] | <0.001 |
| Multivariable analysis | | HR [95% CI] | p-value |
| POLAR | | 1.7 [1.2,2.3] | <0.001 |
| Age | | 0.98 [0.95, 1.0] | 0.1 |
| Histological | 1 | Reference | |
| grade | | | |
| | 2 | 1.1 [0.43, 2.9] | 0.82 |
| | 3 | 0.74 [0.23, 2.4] | 0.61 |
| Tumor size | ≤20 mm | Reference | |
| | >20 mm, | 0.41 [0.055, 3.1] | 0.39 |
| | ≤50 mm | | |
| Subtype | Luminal A | Reference | |
| | Luminal B | 1.5 [0.75, 2.8] | 0.27 |

TABLE 7

| | No RT | RT | Total | p- |
|---|---|---|---|---|
| N (%) | N = 62 | N = 70 | N = 132 | value |
| Age at surgery | | | | |
| (years) | | | | |
| 50-59 | 16 (25.8) | 14 (20) | 30 (22.7) | 0.76 |
| 60-69 | 23 (37.1) | 24 (34.3) | 47 (35.6) | |
| 70-79 | 19 (30.6) | 27 (38.6) | 46 (34.8) | |
| ≥80 | 4 (6.5) | 5 (7.1) | 9 (6.8) | |
| Histological grade | | | | |
| I | 9 (14.5) | 9 (12.9) | 18 (13.6) | 0.43 |
| II | 34 (54.8) | 45 (64.3) | 79 (59.8) | |
| III | 17 (27.4) | 13 (18.6) | 30 (22.7) | |
| NA | 2 (3.2) | 3 (4.3) | 5 (3.8) | |
| Tumor size (mm) | | | | |
| ≤10 | 15 (24.2) | 9 (12.9) | 24 (18.2) | 0.41 |
| 11-20 | 36 (58.1) | 46 (65.7) | 82 (62.1) | |
| 21-30 | 10 (16.1) | 14 (20) | 24 (18.2) | |
| ≥31 | 1 (1.6) | 1 (1.4) | 2 (1.5) | |
| ER status | | | | |
| Positive | 62 (100) | 70 (100) | 132 (100) | |
| PR status | | | | |
| Negative | 7 (11.3) | 10 (14.3) | 17 (12.9) | 0.77 |
| Positive | 49 (79.0) | 52 (74.3) | 101 (76.5) | |
| NA | 6 (9.7) | 8 (11.4) | 14 (10.6) | |
| HER2 status | | | | |
| Negative | 62 (100) | 70 (100) | 132 (100) | |

TABLE 8

| | | | Entrez |
|---|---|---|---|
| Gene | | Coefficient | Gene Id |
| Abbreviation | Gene Name | Value | Number |
| AGR2 | Anterior Gradient 2, Protein | 0.200353 | 10551 |
| | Disulphide Isomerase | | |
| B4GALT1 | Beta-1,4-Galactosyltransferase 1 | −0.33626 | 2683 |
| CLDN7 | Claudin 7 | 0.115625 | 1366 |
| EZR | Ezrin | 0.003256 | 7430 |
| GNG11 | G Protein Subunit Gamma 11 | −0.41974 | 2791 |
| JUN | Jun Proto-Oncogene | −0.01851 | 3725 |
| MMP11 | Matrix Metallopeptidase 11 | 0.444806 | 4320 |
| PKIB | CAMP-Dependent Protein Kinase | 0.170331 | 5570 |
| | Inhibitor Beta | | |
| PRPS1 | Phosphoribosyl Pyrophosphate | 0.170411 | 5631 |
| | Synthetase 1 | | |
| PSMD10 | Proteasome 26S Subunit, Non-ATPase 10 | 0.042903 | 5716 |
| SH3BP5 | SH3 Domain Binding Protein 5 | −0.17135 | 9467 |
| SLC16A3 | Solute Carrier Family 16 Member 3 | 0.214877 | 9123 |
| SLC7A11 | Solute Carrier Family 7 Member 11 | 0.292485 | 23657 |
| SPP1 | Secreted Phosphoprotein 1 | 0.183821 | 6696 |
| TNNT1 | Troponin T1, Slow Skeletal Type | 0.396711 | 7138 |
| UBE2E1 | Ubiquitin Conjugating Enzyme E2 E1 | 0.033286 | 7324 |

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

### INCORPORATION BY REFERENCE

The entire disclosure of each of the patent documents and scientific articles referred to herein is incorporated by reference for all purposes.

What is claimed is:

1. A method, comprising:
a) measuring an expression level of one or more genes in a biological sample from a human patient having or at risk of having breast cancer (BC), wherein the one or more genes are selected from AGR2, B4GALT1, CLDN7, EZR, GNG11, JUN, MMP11, PKIB, PRPS1, PSMD10, SH3BP5, SLC16A3, SLC7A11, SPP1, TNNT1, and UBE2E1; and
b) determining a likelihood of BC recurrence for the patient based on the expression level of the one or more genes.

2. The method of claim 1,
wherein increased expression levels of AGR2, CLDN7, EZR, MMP11, PKIB, PRPS1, PSMD10, SLC16A3, SLC7A11, SPP1, TNNT1, and UBE2E1 are each correlated with an increased risk or likelihood of a breast cancer recurrence;
wherein increased expression levels of B4GALT1, GNG11, JUN, and SH3BP5 are each correlated with a decreased risk or likelihood of a breast cancer recurrence;

3. The method of claim 1,
further comprising treating the patient with adjuvant radiotherapy if the patient is characterized as at high risk for BC recurrence; or
further comprising not treating the patient with adjuvant radiotherapy treatment if the patient is characterized as at low risk for BC recurrence.

4. The method of claim 1, wherein the BC recurrence is local or locoregional recurrence or distant recurrence (metastasis).

5. The method of claim 1, wherein the biological sample is a biopsy or a tumor sample.

6. The method of claim 1, wherein the measuring the levels of expression comprises performing one or more of: in situ hybridization, a PCR-based method, an array-based method, an immunohistochemical method, an RNA assay method, or an immunoassay method.

7. The method of claim 6, wherein the measuring the levels of expression comprises using a reagent selected from the group consisting of a nucleic acid probe, one or more nucleic acid primers, and an antibody.

8. The method of claim 1, wherein the measuring the level of expression comprises measuring the level of an RNA transcript.

9. A kit for determining a prognosis of a patient having breast cancer and whether or not to treat the patient with adjuvant radiotherapy, the kit comprising agents for measuring levels of expression of one or more genes selected from AGR2, B4GALT1, CLDN7, EZR, GNG11, JUN, MMP11, PKIB, PRPS1, PSMD10, SH3BP5, SLC16A3, SLC7A11, SPP1, TNNT1, and UBE2E1.

10. The kit of claim 9, wherein the kit comprises agents for measuring the levels of expression of all of the one or more genes.

11. The kit of claim 10, wherein said agents comprise reagents for performing in situ hybridization, a PCR-based method, an array-based method, an immunohistochemical method, an RNA assay method, or an immunoassay method.

12. The kit of claim 10, wherein said agents comprise one or more of a microarray, a nucleic acid probe, a nucleic acid primer, or an antibody.

13. The kit of claim 10, wherein the kit comprises at least one set of PCR primers capable of amplifying a nucleic acid comprising a sequence of the one or more genes.

14. The kit of claim 10, wherein the kit comprises at least one probe capable of hybridizing to a nucleic acid comprising a sequence of the one or more genes.

15. The kit of claim 10, further comprising information, in electronic or paper form, comprising instructions on how to determine the prognosis of a subject having breast cancer and whether or not to treat the subject with adjuvant radiotherapy.

16. The kit of claim 10, further comprising one or more control reference samples.

17. A probe set for determining a prognosis of a subject having breast cancer and whether or not to treat the subject with adjuvant radiotherapy, the probe set comprising a plurality of probes for detecting a plurality of target nucleic acids, wherein the plurality of target nucleic acids comprises one or more gene sequences selected from AGR2, B4GALT1, CLDN7, EZR, GNG11, JUN, MMP11, PKIB, PRPS1, PSMD10, SH3BP5, SLC16A3, SLC7A11, SPP1, TNNT1, and UBE2E1.

18. The probe set of claim 17, wherein the probe set comprises a plurality of probes for detecting a plurality of target nucleic acids comprising gene sequences, or complements thereof, of the one or more genes.

19. The probe set of claim 17, wherein at least one probe is detectably labeled.

20. A kit for determining a prognosis of a subject having breast cancer and whether or not to treat the subject with adjuvant radiotherapy, chemotherapy, or endocrine therapy, the kit comprising the probe set of claim 17.

21. A system comprising:
a) the probe set of claim 17; and
b) a computer model or algorithm for analyzing an expression level or expression profile of the plurality of target nucleic acids hybridized to the plurality of probes in a biological sample from the subject who has breast cancer and determining if the subject is at low risk of cancer recurrence based on the expression level or expression profile and predicting whether the subject will benefit from adjuvant radiotherapy.

22. A method for generating a risk score to assess a patient's risk for BC recurrence, the method comprising the steps of
identifying a gene expression level for one or more genes present in a sample obtained from the BC patient, wherein the one or more genes is selected from AGR2, B4GALT1, CLDN7, EZR, GNG11, JUN, MMP11, PKIB, PRPS1, PSMD10, SH3BP5, SLC16A3, SLC7A11, SPP1, TNNT1, and UBE2E1;
obtaining a weighted expression level by multiplying the value of said expression level by the coefficient value corresponding to the selected one or more genes from Table 8; and
generating a risk score by adding the weighted expression levels of the one or more genes.

23. The method of claim 22, further comprising assessing the patient's risk for BC recurrence based on the risk score.

24. A method for predicting a likelihood of recurrence of BC for a patient with BC or at risk for having BC comprising:
(a) measuring, in a sample obtained from the patient, an expression level of one or more of the following genes: AGR2, B4GALT1, CLDN7, EZR, GNG11, JUN, MMP11, PKIB, PRPS1, PSMD10, SH3BP5, SLC16A3, SLC7A11, SPP1, TNNT1, and UBE2E1; and
(b) predicting a likelihood of recurrence of BC for the patient based on the expression level of the one or more genes, wherein increased expression of AGR2, CLDN7, EZR, MMP11, PKIB, PRPS1, PSMD10, SLC16A3, SLC7A11, SPP1, TNNT1, and UBE2E1 is correlated with an increased risk of a recurrence of BC, and wherein increased expression of B4GALT1, GNG11, JUN, and SH3BP5 is correlated with a reduced risk of a recurrence of breast cancer.

25. The method of claim 24,
further comprising treating the patient with adjuvant radiotherapy if the patient is characterized as at high risk for BC recurrence; or
further comprising not treating the patient with adjuvant radiotherapy treatment if the patient is characterized as at low risk for BC recurrence.

26. The method of claim 24, wherein the BC recurrence is local or locoregional recurrence or distant recurrence (metastasis).

27. The method of claim 24, wherein the sample is a biopsy or a tumor sample.

28. The method of claim 24, wherein the measuring the levels of expression comprises performing one or more of: in situ hybridization, a PCR-based method, an array-based method, an immunohistochemical method, an RNA assay method, or an immunoassay method.

29. The method of claim 28, wherein the measuring the levels of expression comprises using a reagent selected from the group consisting of a nucleic acid probe, one or more nucleic acid primers, and an antibody.

30. The method of claim 24, wherein the measuring the level of expression comprises measuring the level of an RNA transcript.
